(19) United States
(12) Patent Application Publication
(10) Pub. No.: US 2014/0234217 A1
(43) Pub. Date: Aug. 21, 2014

(54) REMOTE ASSEMBLY OF TARGETED NANOPARTICLES USING COMPLEMENTARY OLIGONUCLEOTIDE LINKERS

(71) Applicant: Mallinckrodt LLC, Hazelwood, MO (US)

(72) Inventor: Thomas Edward Rogers, Ballwin, MO (US)

(21) Appl. No.: 14/348,522

(22) PCT Filed: Sep. 27, 2012
(86) PCT No.: PCT/US2012/057642

§ 371 Date: Mar. 28, 2014

Related U.S. Application Data
(60) Provisional application No. 61/541,797, filed on Sep. 30, 2011.

(51) Int. Cl. A61K 47/48; A61K 49/00; A61K 51/06

(52) U.S. Cl. CPC A61K 47/48169; A61K 47/48815; A61K 51/065; A61K 49/0002
USPC 424/1.73; 536/23.1; 514/44 R; 424/450; 424/451; 424/9.6; 424/9.1

(57) ABSTRACT

The present invention provides targeted delivery compositions and their methods of use in treating and diagnosing a disease state in a subject. Components of the targeted delivery compositions are put together through duplex formation between oligonucleotides.

### CROSS-REFERENCES TO RELATED APPLICATIONS

This application claims the benefit of U.S. Provisional Application Ser. No. 61/541,797, filed Sep. 30, 2011, the entire content of which is incorporated herein by reference.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Cancer is a class of diseases that can affect people of all ages. Accordingly, there is considerable effort to provide therapies that can treat or diagnose cancer in patients. Targeted delivery of nanoparticles in the body has been discussed recently as a potential new avenue in drug delivery and diagnostic imaging techniques. Unfortunately, obstacles still exist in making nanoparticle based-products that can effectively treat or diagnose cancer. Thus, there is a need for new targeted delivery approaches that can treat or diagnose cancer and provide ways to facilitate personalized care for a patient.

### BRIEF SUMMARY OF THE INVENTION

The present invention provides targeted delivery compositions and their methods of use in treating and diagnosing a disease state, such as a cancerous condition, in a subject.

In an aspect of the invention, the targeted delivery compositions can include a nanoparticle including a therapeutic agent, diagnostic agent, or combination thereof, a derivatized attachment component having the formula: A-(L¹)ₓ-C¹, and a targeting component having the formula: C²-(L²)_{y}-T, each of which is described in more detail below. In another aspect, the targeted delivery compositions can include a diagnostic or therapeutic component having the formula: DT-(L¹)ₓ-C¹, and a targeting component having the formula: C²-(L²)_{y}-T, each of which is described in more detail below.

The targeted delivery compositions and methods of making and using such compositions provide a number of unique aspects to the areas of drug delivery and diagnostic imaging. For example, certain components (e.g., the nanoparticle and the attachment component) of the targeted delivery compositions can be put together by a variety of processes before the targeting component is added to form a final assembly. Duplex formation techniques as described herein can provide these advantages. In certain instances, these advantages can also be used for providing a more personalized approach for treating and/or diagnosing a condition of subject, e.g., the targeted delivery compositions can provide advancements in personalized medicine approaches.

A further understanding of the nature and advantages of the present invention can be realized by reference to the remaining portions of the specification and the drawings.

### BRIEF DESCRIPTION OF THE DRAWINGS

FIG. 1 illustrates a structure of a targeted delivery composition in accordance with an exemplary embodiment of the invention.

FIG. 2 illustrates a crosslinking reaction in accordance with an exemplary embodiment of the invention.

### DETAILED DESCRIPTION OF THE INVENTION

### I. Definitions

As used herein, the term “targeted delivery composition” refers generally to a composition that can be used to treat and/or diagnose a disease state in a subject. In some embodiments, a targeted delivery composition of the present invention can include “a targeted therapeutic or targeted diagnostic delivery composition” that can include a nanoparticle, a derivatized attachment component, and a targeting component, as described herein. In other embodiments, the targeted delivery compositions of the present invention can include a diagnostic or therapeutic component and a targeting component. The compositions of the present invention can be used as therapeutic compositions, as diagnostic compositions, or as both therapeutic and diagnostic compositions. In certain embodiments, the compositions can be targeted to a specific target within a subject or a test sample, as described further herein.

As used herein, the term “nanoparticle” refers to particles of varied size, shape, type and use, which are further described herein. As will be appreciated by one of ordinary skill in the art, the characteristics of the nanoparticles, e.g., size, can depend on the type and/or use of the nanoparticle as well as other factors generally well known in the art. In general, nanoparticles can range in size from about 1 nm to about 1000 nm. In other embodiments, nanoparticles can range in size from about 10 nm to about 200 nm. In yet other embodiments, nanoparticles can range in size from about 50 nm to about 150 nm. In certain embodiments, the nanoparticles are greater in size than the renal excretion limit, e.g., greater than about 6 nm in diameter. In other embodiments, the nanoparticles are small enough to avoid clearance from the bloodstream by the liver, e.g., smaller than 1000 nm in diameter. Nanoparticles can include spheres, cones, spheroids, and other shapes generally known in the art. Nanoparticles can be hollow (e.g., solid outer core with a hollow inner core) or solid or be multilayered with hollow and solid layers or a variety of solid layers. For example, a nanoparticle can include a solid core region and a solid outer encapsulating region, both of which can be cross-linked. Nanoparticles can be composed of one substance or any combination of a variety of substances, including lipids, polymers, magnetic materials, or metallic materials, such as silica, iron oxide, and the like. Lipids can include fats, waxes, sterols, cholesterol, a cholesterol derivative, fat-soluble vitamins, monoglycerides, diglycerides, phospholipids, sphingolipids, glycolipids, cationic or anionic lipids, derivatized lipids, cardiolipin and the like. Polymers can include block copolymers generally, poly(lactic acid), poly(lactic-co-glycolic acid), polyethylene glycol, acrylic polymers, cationic polymers, as well as other polymers known in the art for use in making nanoparticles. In some embodiments, the polymers can be biodegradable and/or biocompatible. Nanoparticles can include a liposome, a micelle, a lipoprotein, a lipid-coated bubble, a block copolymer micelle, a polymersome, a niosome, a quantum dot, an iron oxide particle, a dendrimer, or a silica particle. In certain embodiments, a lipid monolayer or bilayer can fully or partially coat a nanoparticle composed of a material capable of being coated by lipids, e.g., polymer nanoparticles. In some embodiments, liposomes can include multilamellar vesicles (MLV), large unilamellar vesicles (LUV), and small unilamellar vesicles (SUV).

As used herein, the term “therapeutic agent” refers to a compound or molecule that, when present in an effective amount, produces a desired therapeutic effect on a subject in need thereof. The present invention contemplates a broad range of therapeutic agents and their use in conjunction with the targeted delivery compositions, as further described herein.

As used herein, the term “diagnostic agent” refers to a component that can be detected in a subject or test sample and is further described herein.

As used herein, the term “attachment component” refers to the A portion of the derivatized attachment component having the formula A-(L¹)ₓ-C¹, as described further herein. The attachment component of the present invention can attach (covalently or non-covalently) to a nanoparticle. In certain embodiments, an attachment component can be covalently bonded to any part of a nanoparticle including the surface or an internal region. Covalent attachment can be achieved using a linking chemistry known generally in the art, including but not limited to that which is further described herein. In other embodiments, a non-covalent interaction can include affinity interactions, metal coordination, physical adsorption, hydrophobic interactions, van der Waals interactions, hydrogen bonding interactions, magnetic interactions, electrostatic interactions, dipole-dipole interactions, antibody-binding interactions, and the like. In some embodiments, an attachment component can be present in a lipid bilayer portion of a nanoparticle, wherein in certain embodiments the nanoparticle is a liposome. For example, an attachment component can be a lipid that interacts partially or wholly with the hydrophobic and/or hydrophilic regions of the lipid bilayer.

As used herein, the term “derivatized” refers to a derivative form of a molecule, which is modified or made suitable for a particular purpose. For example, a derivatized attachment component of the present invention can have the formula A-(L¹)-C¹, such that the attachment component is derivatized with a hydrophilic, non-immunogenic, water soluble linking group which can in turn be covalently attached to an oligonucleotide, e.g., C¹.

As used herein, the term “targeting component” refers to a component of the targeted delivery compositions having the formula C²-(L²)_{y}-T, as described further herein. In certain embodiments, the targeting components of the present invention can bind to a specific target, e.g., a target on a cancer cell, an epitope, a tissue site or receptor site.

As used herein, the term “targeting agent” refers to a molecule that is specific for a target. In certain embodiments, a targeting agent can include a small molecule mimic of a target ligand (e.g., a peptide mimetic ligand), a target ligand (e.g., an RGD peptide containing peptide or folate amide), or an antibody or antibody fragment specific for a particular target. Targeting agents can bind a wide variety of targets, including targets in organs, tissues, cells, extracellular matrix components, and/or intracellular compartments that can be associated with a specific developmental stage of a disease. In some embodiments, targets can include cancer cells, particularly cancer stem cells. Targets can further include antigens on a surface of a cell, or a tumor marker that is an antigen present or more prevalent on a cancer cell as compared to normal tissue. In certain embodiments, a targeting agent can further include folic acid derivatives, B-12 derivatives, integrin RGD peptides, RGD mimetics, NGR derivatives, somatostatin derivatives or peptides that bind to the somatostatin receptor, e.g., octreotide and octreotate, and the like. In some embodiments, a targeting agent can be an aptamer—which is composed of nucleic acids (e.g., DNA or RNA), or a peptide and which binds to a specific target. A targeting agent can be designed to bind specifically or non-specifically to receptor targets, particularly receptor targets that are expressed in association with tumors. Examples of receptor targets include, but are not limited to, MUC-1, EGFR, Claudin 4, MUC-4, CXCR4, CCR7, FOL1R, somatostatin receptor 4, Erb-B2 (erythroblastic leukaemia oncogene homologue 2) receptor, CD44 receptor, and VEGF receptor-2 kinase.

As used herein, the term “hydrophilic, non-immunogenic, water soluble linking group” refers to a molecule linking one portion of a component to another portion of the same component. The linking groups are further described herein and include L¹ and L².

As used herein, the term “oligonucleotide” refers generally to a chain of nucleotides that can include any nucleotide chain of more than one nucleotide. An oligonucleotide can, for example, include short nucleotide sequences from 8 to 20 nucleic acids. In some embodiments, oligonucleotides can range from about 2 to about 100 nucleic acids in length, from about 2 to about 50 nucleic acids in length, from about 8 to about 50 nucleic acids in length, from about 8 to about 40 nucleic acids in length, from about 10 to about 30 nucleic acids in length, or from about 20 to about 30 nucleic acids in length. An oligonucleotide can, e.g., include natural bases (e.g., adenine, guanine, thymine, uracil, and cytosine). In some embodiments, the oligonucleotide sequence can be natural or non-natural. In certain embodiments, oligonucleotides can form duplexes and can be either DNA or RNA.

As used herein, the term “oligonucleotide mimic” refers to molecules that can mimic DNA or RNA. Oligonucleotide mimics can include artificial or non-natural mimics, such as peptide nucleic acids (PNA) and other phosphorothioate analogs. In some embodiments, the oligonucleotide mimics can form duplexes together (e.g., PNA/PNA) or with oligonucleotides (e.g., PNA/DNA or PNA/RNA). In certain embodiments, universal and/or modified bases can be used.

As used herein, the term “linking moiety” refers to a chemical group capable of linking two or more oligonucleotides together typically by covalent attachment. For example, in certain embodiments of the present invention nucleotide pairs can be cross-linked together under certain conditions, such as photocrosslinking, or base or acid catalyzed cross-linking. Methods for cross-linking between or among oligonucleotides are well known and, for example, are described in Webb, Thomas R., Matteucci, Mark D., Nucleic Acids Research (1986) 14(19), 7661-7674.

As used herein, the term “stealth agent” refers to a molecule that can modify the surface properties of a nanoparticle and is further described herein.

As used herein, the term “embedded in” refers to the location of an agent on or in the vicinity of the surface of a nanoparticle. Agents embedded in a nanoparticle can, for example, be located within a bilayer membrane of a liposome or located within an outer polymer shell of a nanoparticle so as to be contained within that shell.

As used herein, the term “encapsulated in” refers to the location of an agent that is enclosed or completely contained within the inside of a nanoparticle. For liposomes, for example, therapeutic and/or diagnostic agents can be encapsulated so as to be present in the aqueous interior of the liposome. Release of such encapsulated agents can then be triggered by certain conditions intended to destabilize the liposome or otherwise effect release of the encapsulated agents.

As used herein, the term “tethered to” refers to attachment of one component to another component so that one or more of the components has freedom to move about in space. In certain exemplary embodiments, an attachment component can be tethered to a nanoparticle so as to freely move about in solution surrounding the nanoparticle. In some embodiments, an attachment component can be tethered to the surface of a nanoparticle, extending away from the surface.

As used herein, the term “functional group for covalent attachment” refers to a portion of a first molecule that can be used to covalently attach the first molecule to another functional group on a second molecule (or another site on the first molecule). Functional groups are well known in the art and can include without limitation amino, hydroxyl, carboxylic acid, amide, azides, α-haloketones, α,β-unsaturated ketones, alkynes, dienes, enamines, maleimido groups, thiols, and the like.

As used herein, the term “lipid” refers to lipid molecules that can include fats, waxes, sterols, cholesterol, a cholesterol derivative, fat-soluble vitamins, monoglycerides, diglycerides, phospholipids, sphingolipids, glycolipids, cationic or anionic lipids, derivatized lipids, and the like. Lipids can form micelles, monolayers, and bilayer membranes. In certain embodiments, the lipids can self-assemble into liposomes. In other embodiments, the lipids can coat a surface of a nanoparticle as a monolayer or a bilayer.

As used herein, the term “aptamer” refers to a nucleic acid or peptide molecule that binds to a specific target. DNA or RNA aptamers can include but are not limited to short oligonucleotide sequences that can be natural or non-natural and can be selected using in vitro selection processes, such as SELEX (systematic evolution of ligands by exponential enrichment). SELEX is described, for example, in U.S. Pat. Nos. 5,270,163 and 5,475,096, which are incorporated by reference herein. Other selection processes can further include MonoLex™ technology (single round aptamer isolation procedure of AptaRes AG; described, e.g., in US Publication No. 20090269752), in vivo selection processes, or combinations thereof. Aptamers for use in the present invention can be designed to bind to a variety of targets, including but not limited to MUC-1, EGFR, Claudin 4, MUC-4, CXCR4, CCR7, FOL1R, somatostatin receptor 4, Erb-B2 (erythroblastic leukaemia oncogene homologue 2) receptor, CD44 receptor, VEGF receptor-2 kinase, and nucleolin.

As used herein, the term “preferential binding pair” refers to a pair of molecules that bind to each other, e.g., oligonucleotides or oligonucleotide mimics, typically in a specific manner. In certain embodiments, a preferential binding pair can include one oligonucleotide member that has a preference for binding to a single or a plurality of DNA sequences over others, e.g., a second oligonucleotide member. For a given oligonucleotide, there are a spectrum of differential affinities for different DNA sequences ranging from non-sequence-specific (no detectable preference) to sequence preferential to absolute sequence specificity (i.e., the recognition of only a single sequence among all possible sequences). The preferential nature of a binding pair can be described in a variety of ways, such as by melting temperature or complementarity between the two binding pair members. In certain embodiments, the preferential binding pair includes C¹ and C², as further described herein.

As used herein, the term “complementary” refers to an amount of base pairing between oligonucleotide strands. In certain embodiments, the amount of complementarity between two oligonucleotides can be expressed in percentages. For example, a first oligonucleotide strand is fully complementary (i.e., 100% complementary) to a second oligonucleotide strand if base pairing is formed between each contiguous nucleotide along the first and second oligonucleotide strands. In some embodiments, the full length or a portion of the length of an oligonucleotide strand will be complementary (e.g., fully complementary) to another oligonucleotide strand. Complementary oligonucleotide strands can be a different length or the same length. In certain embodiments, the oligonucleotides of the present invention can be at least 70% complementary. For example, two oligonucleotides that are 70% complementary can have a length of, e.g., ten nucleotides, in which seven of the oligonucleotides form base pairs and three do not. In other embodiments, the oligonucleotides can be greater than 80% complementary, or greater than 90% complementary, or greater than 95% complementary. The term “percent identity” can also be used in the context of two or more nucleic acids or polypeptide sequences that are the same or have a specified percentage of nucleotides or amino acid residues that are the same, when compared and aligned for maximum correspondence. To determine the percent identity, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100).

As used herein, the term “conditions sufficient for a duplex to be formed” refers to conditions that allow for oligonucleotide hybridization. Hybridization of an oligonucleotide and another oligonucleotide can be accomplished by choosing appropriate hybridization conditions. In certain embodiments, hybridization conditions can include conditions sufficient to form a duplex between oligonucleotides or oligonucleotide mimics. For example, the stability of the oligonucleotide:oligonucleotide hybrid is typically selected to be compatible with the assay and washing conditions so that stable, detectable hybrids form only between the specific oligonucleotides. Manipulation of one or more of the different assay parameters determines the exact sensitivity and specificity of a particular hybridization assay. More specifically, hybridization between complementary bases of DNA, RNA, PNA, or combinations of DNA, RNA and PNA, occurs under a wide variety of conditions that vary in temperature, salt concentration, electrostatic strength, buffer composition, and the like. Examples of these conditions and methods for applying them are described in, e.g., Tijssen, Hybridization with Nucleic Acid Probes, Vol. 24, Elsevier Science (1993). Hybridization generally takes place between about 0° C. and about 70° C., for periods of from about one minute to about one hour, depending on the nature of the sequence to be hybridized and its length. However, it is recognized that hybridizations can occur in seconds or hours, depending on the conditions of the reaction.

As used herein, the term “non-natural” refers to sequences or molecules that do not naturally occur in nature. Non-natural sequences can be used to provide specific binding only between two preferential binding pairs, so as to not allow binding with other naturally-occurring oligonucleotide sequences present in a test sample or a subject receiving treatment.

As used herein, the term “subject” refers to any mammal, in particular human, at any stage of life.

As used herein, the terms “administer,” “administered,” or “administering” refers to methods of administering the targeted delivery compositions of the present invention. The targeted delivery compositions of the present invention can be administered in a variety of ways, including topically, parenterally, intravenously, intradermally, intramuscularly, colonically, rectally or intraperitoneally. Parenteral administration, oral administration, and intravenous administration are the preferred methods of administration. The targeted delivery compositions can also be administered as part of a composition or formulation.

As used herein, the terms “treating” or “treatment” of a condition, disease, disorder, or syndrome includes (i) inhibiting the disease, disorder, or syndrome, i.e., arresting its development; and (ii) relieving the disease, disorder, or syndrome, i.e., causing regression of the disease, disorder, or syndrome. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

As used herein, the term “formulation” refers to a mixture of components for administration to a subject. Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can also be prepared from sterile powders, granules, and tablets. The formulations of a targeted delivery composition can be presented in unit-dose or multi-dose sealed containers, such as ampoules and vials. A targeted delivery composition, alone or in combination with other suitable components, can be made into aerosol formulations (i.e., they can be “nebulized”) to be administered via inhalation through the mouth or the nose. Aerosol formulations can be placed into pressurized acceptable propellants, such as dichlorodifluoromethane, propane, nitrogen, and the like. Suitable formulations for rectal administration include, for example, suppositories, which comprises an effective amount of a targeted delivery composition with a suppository base. Suitable suppository bases include natural or synthetic triglycerides or paraffin hydrocarbons. In addition, it is also possible to use gelatin rectal capsules which contain a combination of the targeted delivery composition with a base, including, for example, liquid triglycerides, polyethylene glycols, and paraffin hydrocarbons. In certain embodiments, formulations can be administered topically or in the form of eye drops.

### Embodiments of the Invention

### II. General

The present invention provides targeted delivery compositions and their methods of use in treating and diagnosing a disease state in a subject. The disclosed compositions and methods provide a number of beneficial features over currently existing approaches. For example, the targeted delivery compositions and methods of the invention can be used for personalized medicine approaches that can treat and/or diagnose a disease state in a subject. For example, the duplex linkage between components of the targeted delivery compositions provides unique advantages that allow for additional freedom in defining how to assemble the targeting delivery compositions.

### III. Targeted Delivery Compositions

### A. Targeted Delivery Compositions Including a Nanoparticle

In one aspect, the targeted delivery compositions of the present invention can include a targeted therapeutic or diagnostic delivery composition, comprising (a) a nanoparticle including a therapeutic agent or a diagnostic agent or a combination thereof; (b) a derivatized attachment component having the formula: A-(L¹)ₓ-C¹; and (c) a targeting component having the formula: C²-(L²)_{y}-T, wherein, A is an attachment component; each of L¹ and L² is a hydrophilic, non-immunogenic, water soluble linking group; C¹ is one member of a preferential binding pair with a second member C², wherein C¹ and C² are oligonucleotides or oligonucleotide mimics; T is a targeting agent; and each of the subscripts x and y are independently 0 or 1, but at least one of x and y is other than 0; wherein the A portion of the derivatized attachment component is attached to the nanoparticle.

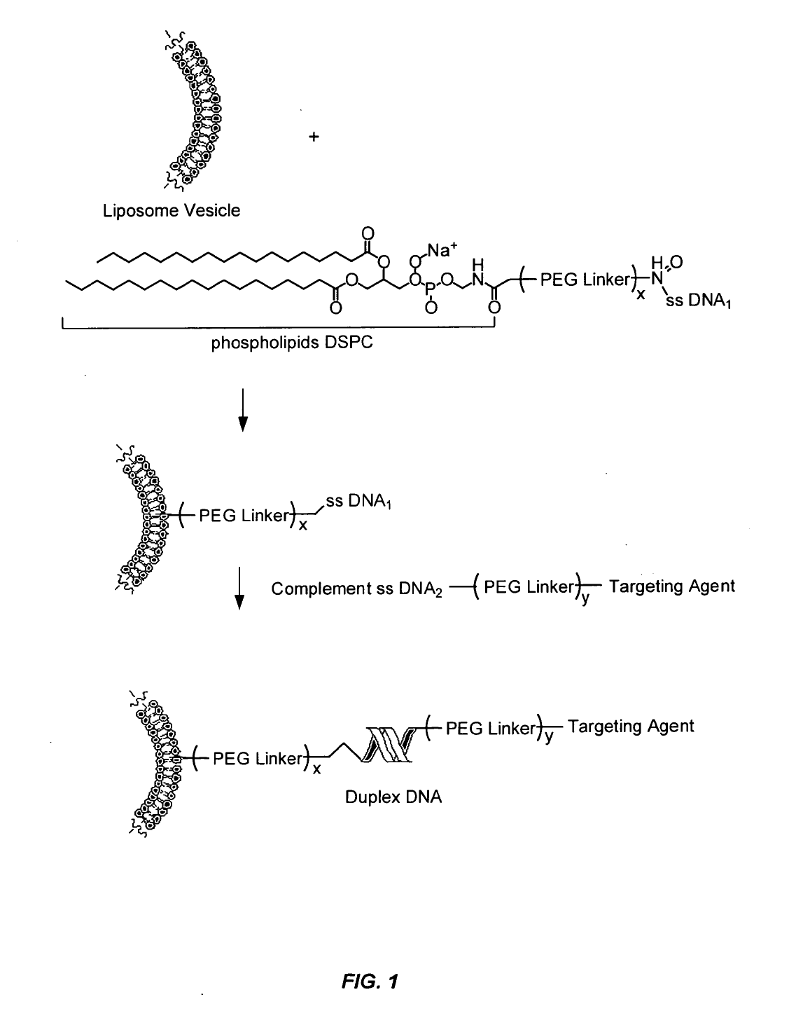

FIG. 1 illustrates a general structure of a targeted delivery composition in accordance with an exemplary embodiment of the invention. A portion of a liposome is provided showing a lipid bilayer membrane. A derivatized attachment component can be composed of a lipid attachment component, A, which is 1,2-distearoyl-sn-glycero-3-phosphoethanolamine (DSPE). The lipid attachment component can be covalently attached to a polyethylene glycol (PEG) linker, which can be covalently attached to a single strand of DNA (C¹). A targeting component can be composed of a single strand of DNA (C₂) that is complementary to C¹ and covalently attached to a PEG linker, which is further covalently attached to a targeting agent. A targeted delivery composition can be composed of the derivatized attachment component in which the lipid end is associated with the lipid bilayer of the liposome and the single strand DNA, C₂, of the targeting agent hybridizes with the single strand DNA, C¹, of the derivatized attachment component.

### Nanoparticles

A wide variety of nanoparticles can be used in constructing the targeted delivery compositions. As will be appreciated by one of ordinary skill in the art, the characteristics of the nanoparticles, e.g., size, can depend on the type and/or use of the nanoparticle as well as other factors generally well known in the art. Suitable particles can be spheres, spheroids, flat, plate-shaped, tubes, cubes, cuboids, ovals, ellipses, cylinders, cones, or pyramids. Suitable nanoparticles can range in size of greatest dimension (e.g., diameter) from about 1 nm to about 1000 nm, from about 50 nm to about 200 nm, and from about 50 nm to about 150 nm.

Suitable nanoparticles can be made of a variety of materials generally known in the art. In some embodiments, nanoparticles can include one substance or any combination of a variety of substances, including lipids, polymers, or metallic materials, such as silica, iron oxide, and the like. Examples of nanoparticles can include but are not limited to a liposome, a micelle, a lipoprotein, a lipid-coated bubble, a block copolymer micelle, a polymersome, a niosome, an iron oxide particle, a silica particle, a dendrimer, or a quantum dot.

In some embodiments, the nanoparticles are liposomes composed partially or wholly of saturated or unsaturated lipids. Suitable lipids can include but are not limited to fats, waxes, sterols, cholesterol, a cholesterol derivative, fat-soluble vitamins, monoglycerides, diglycerides, phospholipids, sphingolipids, glycolipids, derivatized lipids, and the like. In some embodiments, suitable lipids can include amphipathic, neutral, non-cationic, anionic, cationic, or hydrophobic lipids. In certain embodiments, lipids can include those typically present in cellular membranes, such as phospholipids and/or sphingolipids. Suitable phospholipids include but are not limited to phosphatidylcholine (PC), phosphatidic acid (PA), phosphatidylethanolamine (PE), phosphatidylglycerol (PG), phosphatidylserine (PS), and phosphatidylinositol (PI). Suitable sphingolipids include but are not limited to sphingosine, ceramide, sphingomyelin, cerebrosides, sulfatides, gangliosides, and phytosphingosine. Other suitable lipids can include lipid extracts, such as egg PC, heart extract, brain extract, liver extract, and soy PC. In some embodiments, soy PC can include Hydro Soy PC(HSPC). Cationic lipids include but are not limited to N,N-dioleoyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), and N,N-dimethyl-2,3-dioleyloxy)propylamine (DODMA). Non-cationic lipids include but are not limited to dimyristoyl phosphatidyl choline (DMPC), distearoyl phosphatidyl choline (DSPC), dioleoyl phosphatidyl choline (DOPC), dipalmitoyl phosphatidyl choline (DPPC), dimyristoyl phosphatidyl glycerol (DMPG), distearoyl phosphatidyl glycerol (DSPG), dioleoyl phosphatidyl glycerol (DOPG), dipalmitoyl phosphatidyl glycerol (DPPG), dimyristoyl phosphatidyl serine (DMPS), distearoyl phosphatidyl serine (DSPS), dioleoyl phosphatidyl serine (DOPS), dipalmitoyl phosphatidyl serine (DPPS), dioleoyl phosphatidyl ethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), 1,2-dielaidoyl-sn-glycero-3-phophoethanolamine (transDOPE), and cardiolipin. In certain embodiments, the lipids can include derivatized lipids, such as PEGlyated lipids. Derivatized lipids can include, for example, DSPE-PEG2000, cholesterol-PEG2000, DSPE-polyglycerol, or other derivatives generally well known in the art.

Any combination of lipids can be used to construct a nanoparticle, such as a liposome. In certain embodiments, the lipid composition of a targeted delivery composition, such as a liposome, can be tailored to affect characteristics of the liposomes, such as leakage rates, stability, particle size, zeta potential, protein binding, in vivo circulation, and/or accumulation in tissue, such as a tumor, liver, spleen or the like. For example, DSPC and/or cholesterol can be used to decrease leakage from the liposomes. Negatively or positively lipids, such as DSPG and/or DOTAP, can be included to affect the surface charge of a liposome. In some embodiments, the liposomes can include about ten or fewer types of lipids, or about five or fewer types of lipids, or about three or fewer types of lipids. In some embodiments, the molar percentage (mol %) of a specific type of lipid present typically comprises from about 0% to about 10%, from about 10% to about 30%, from about 30% to about 50%, from about 50% to about 70%, from about 70% to about 90%, from about 90% to 100% of the total lipid present in a nanoparticle, such as a liposome. The lipids described herein can be included in a liposome, or the lipids can be used to coat a nanoparticle of the invention, such as a polymer nanoparticle. Coatings can be partially or wholly surrounding a nanoparticle and can include monolayers and/or bilayers. In one embodiment, liposomes can be composed of about 50.6 mol % HSPC, about 44.3 mol % cholesterol, and about 5.1 mol % DSPE-PEG2000.

In other embodiments, a portion or all of a nanoparticle can include a polymer, such as a block copolymer or other polymers known in the art for making nanoparticles. In some embodiments, the polymers can be biodegradable and/or biocompatible. Suitable polymers can include but are not limited to polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, and combinations thereof. In some embodiments, exemplary particles can include shell cross-linked knedels, which are further described in the following references: Becker et al., U.S. application Ser. No. 11/250,830; Thurmond, K. B. et al., J. Am. Chem. Soc., 119 (28) 6656-6665 (1997)); Wooley, K. L., Chem. Eur. J., 3 (9): 1397-1399 (1997); Wooley, K. L., J. Poly. Sci.: Part A: Polymer Chem., 38: 1397-1407 (2000). In other embodiments, suitable particles can include poly(lactic co-glycolic acid) (PLGA) (Fu, K. et al., Pharm Res., 27:100-106 (2000).

In yet other embodiments, the nanoparticles can be partially or wholly composed of materials that are metallic in nature, such as silica, iron oxide, and the like. In some embodiments, the silica particles can be hollow, porous, and/or mesoporous (Slowing, L¹., et al., Adv. Drug Deliv. Rev., 60 (11):1278-1288 (2008)). Iron oxide particles or quantum dots can also be used and are well-known in the art (van Vlerken, L. E. & Amiji, M. M., Expert Opin. Drug Deliv., 3(2): 205-216 (2006)). The nanoparticles also include but are not limited to viral particles and ceramic particles.

### Derivatized Attachment Components

In certain embodiments, the targeted delivery compositions of the present invention also can include a derivatized attachment component having the formula: A-(L¹)ₓ-C¹. The attachment component A can be used to attach the derivatized attachment component to a nanoparticle. The attachment component can attach to any location on the nanoparticle, such as on the surface of the nanoparticle. The attachment component can attach to the nanoparticle through a variety of ways, including covalent and/or non-covalent attachment. As described further below, the derivatized attachment component also includes a linking group, L¹, and a member of a preferential binding pair, C¹.

In certain embodiments, the attachment component A can include a functional group that can be used to covalently attach the attachment component to a reactive group present on the nanoparticle. The functional group can be located anywhere on the attachment component, such as the terminal position of the attachment component. A wide variety of functional groups are generally known in the art and can be reacted under several classes of reactions, such as but not limited to nucleophilic substitutions (e.g., reactions of amines and alcohols with acyl halides or active esters), electrophilic substitutions (e.g., enamine reactions) and additions to carbon-carbon and carbon-heteroatom multiple bonds (e.g., Michael reaction or Diels-Alder addition). These and other useful reactions are discussed in, for example, March, Advanced Organic Chemistry, 3rd Ed., John Wiley & Sons, New York, 1985; and Hermanson, Bioconjugate Techniques, Academic Press, San Diego, 1996. Suitable functional groups can include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc. (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides or reacted with acyl halides; (h) amine or sulfhydryl groups, which can be, for example, acylated, alkylated or oxidized; (i) alkenes, which can undergo, for example, cycloadditions, acylation, Michael addition, etc; and (j) epoxides, which can react with, for example, amines and hydroxyl compounds. In some embodiments, click chemistry-based platforms can be used to attach the attachment component to a nanoparticle (Kolb, H. C. et al. M. G. Finn and K. B. Sharpless, Angew. Chem. Int'l. Ed. 40 (11): 2004-2021 (2001)). In some embodiments, the attachment component can include one functional group or a plurality of functional groups that result in a plurality of covalent bonds with the nanoparticle.

Table 1 provides an additional non-limiting, representative list of functional groups that can be used in the present invention.

TABLE 1
Exemplary Functional Group Pairs for Conjugation Chemistry

| Functional Groups: | Reacts with: |
|---|---|
| Ketone and aldehyde groups | Amino, hydrazido and aminooxy |
| Imide | Amino, hydrazido and aminooxy |
| Cyano | Hydroxy |
| Alkylating agents (such as haloalkyl | Thiol, amino, hydrazido, |
| groups and maleimido derivatives) | aminooxy |
| Carboxyl groups (including activated | Amino, hydroxyl, hydrazido, |
| carboxyl groups) | aminooxy |
| Activated sulfonyl groups (such as | Amino, hydroxyl, hydrazido, |
| sulfonyl chlorides) | aminooxy |
| Sulfhydryl | Sulfhydryl |
| His-tag (such as 6-His tagged peptide or | Nickel nitriloacetic acid |
| protein) | |

In other embodiments, an attachment component can be attached to a nanoparticle by non-covalent interactions that can include but are not limited to affinity interactions, metal coordination, physical adsorption, hydrophobic interactions, van der Waals interactions, hydrogen bonding interactions, magnetic interactions, electrostatic interactions, dipole-dipole interactions, antibody-binding interactions, hybridization interactions between complementary DNA, and the like. In some embodiments, an attachment component can be present in a lipid bilayer portion of a nanoparticle, wherein in certain embodiments the nanoparticle is a liposome. For example, an attachment component can be a lipid that interacts partially or wholly with the hydrophobic and/or hydrophilic regions of the lipid bilayer. In some embodiments, the attachment component can include one group that allows non-covalent interaction with the nanoparticle, but a plurality of groups is also contemplated. For example, a plurality of ionic charges can be used to produce sufficient non-covalent interaction between the attachment component and the nanoparticle. In alternative embodiments, the attachment component can include a plurality of lipids such that the plurality of lipids interacts with a bilayer membrane of a liposome or bilayer or monolayer coated on a nanoparticle. In certain embodiments, surrounding solution conditions can be modified to disrupt non-covalent interactions thereby detaching the attachment component from the nanoparticle.

### Linking Groups

Linking groups are another feature of the targeted delivery compositions of the present invention. One of ordinary skill in the art can appreciate that a variety of linking groups are known in the art and can be found, for example, in the following reference: Hermanson, G. T., Bioconjugate Techniques, 2^{nd} Ed., Academic Press, Inc. (2008). Linking groups of the present invention can be used to provide additional properties to the composition, such as providing spacing between different portions of a component. For example, the attachment component can be spaced a distance away from the member of the preferential binding pair (e.g., C¹). This spacing can be used, for example, to facilitate binding between members of the preferential binding pair. Alternatively, additional spacing can be used to overcome steric hindrance issues caused by the nanoparticle, e.g., when a targeting agent binds to a target. In some embodiments, linking groups can be used to change the physical properties of the targeted delivery composition, such as modifying the hydrophilic or hydrophobic nature of a component.

In one group of embodiments, the derivatized attachment component and targeting component can include a hydrophilic, non-immunogenic, water soluble linking group, such as L¹ and L², respectively. For the derivatized attachment component, a hydrophilic, non-immunogenic, water soluble linking group links an attachment component A to a member of a preferential binding pair, e.g., C¹. For the targeting component, a hydrophilic, non-immunogenic, water soluble linking group links a targeting agent to a member of a preferential binding pair, e.g., C². The hydrophilic, non-immunogenic, water soluble linking group can include but is not limited to polyalkylene glycol, polyethylene glycol, polypropylene glycol, polyvinyl alcohol, polycarboxylate, polysaccharide, and dextran. A list of potential linking groups is further described in US Application No. 20090149643. In certain embodiments, a polyethylene glycol (PEG) linking group can include an oligomer or polymer of ethylene oxide. The invention contemplates use of PEG and its derivatives that are generally known in the art. For example, polyethylene glycol linking groups can be linear or branched, wherein branched PEG molecules can have additional PEG molecules emanating from a central core and/or multiple PEG molecules can be grafted to the polymer backbone. Polyethylene glycol linking groups can be derivatized. Polyethylene glycol linking groups can be of low or high molecular weight and can include, e.g., PEG₅₀₀, PEG₂₀₀₀, PEG₃₄₀₀, PEG₅₀₀₀, PEG₁₀₀₀₀, or PEG₂₀₀₀₀ wherein the number, e.g., 500, indicates the average molecular weight. In certain embodiments, the PEG linking groups can include polydisperse and/or monodisperse PEG.

The number of hydrophilic, non-immunogenic, water soluble linking groups present in the derivatized attachment component or the targeting component, such as L¹ and L², can be indicated by subscripts x and y, respectively. In the present invention, various combinations are useful for the linking groups. In some embodiments, each of the subscripts x and y can be independently zero or one. In other embodiments, at least one of x and y is other than zero. In yet other embodiments, x can be zero and y can be one.

### Stealth Agents

In some embodiments, the targeted delivery compositions of the present invention can include at least one stealth agent. A stealth agent can prevent nanoparticles from sticking to each other and to blood cells or vascular walls. In certain embodiments, stealth nanoparticles, e.g., stealth liposomes, can reduce immunogenicity and/or reactogenecity when the nanoparticles are administered to a subject. Stealth agents can also increase blood circulation time of a nanoparticle within a subject. In some embodiments, a nanoparticle can include a stealth agent such that, for example, the nanoparticle is partially or fully composed of a stealth agent or the nanoparticle is coated with a stealth agent. Stealth agents for use in the present invention can include those generally well known in the art. Suitable stealth agents can include but are not limited to dendrimers, polyalkylene oxide, polyethylene glycol, polyvinyl alcohol, polycarboxylate, polysaccharides, and/or hydroxyalkyl starch. Stealth agents can be attached to the phosphonate compounds described herein through covalent and/or non-covalent attachment, as described above with respect to the attachment component. For example, in some embodiments, attachment of the stealth agent to a phosphonate compound described herein can involve a reaction between a terminal functional group (e.g., an amino group) on the stealth agent with a linking group terminated with a functional group (e.g., a carboxyl group).

In certain embodiments, a stealth agent can include a polyalkylene oxide, such as “polyethylene glycol,” which is well known in the art and refers generally to an oligomer or polymer of ethylene oxide. Polyethylene glycol (PEG) can be linear or branched, wherein branched PEG molecules can have additional PEG molecules emanating from a central core and/or multiple PEG molecules can be grafted to the polymer backbone. As is understood in the art, polyethylene glycol can be produced in as a distribution of molecular weights, which can be used to identify the type of PEG. For example, PEG₅₀₀ is identified by a distribution of PEG molecules having an average molecular weight of ˜500 g/mol, as measured by methods generally known in the art. Alternatively, PEG can be represented by the following formula: H—[O—(CH₂)₂]₂—OH, in which n is the number of monomers present in the polymer (e.g., n can range from 1 to 200). For example, for a distribution of PEG₁₀₀ can include PEG polymers in which n is equal to 2. In another instance, PEG₁₀₀₀ can include PEG molecules in which n is equal to 24. Alternatively, PEG₅₀₀₀ can include PEG molecules in which n is equal to 114. In some embodiments, PEG can be terminated by a methyl group instead of an —OH group, as shown above.

In certain embodiments, PEG can include low or high molecular weight PEG, e.g., PEG₁₀₀, PEG₅₀₀, PEG₁₀₀₀, PEG₂₀₀₀, PEG₃₄₀₀, PEG₅₀₀₀, PEG₁₀₀₀₀, or PEG₂₀₀₀₀. In some embodiments, PEG can range between PEG₁₀₀ to PEG₁₀₀₀₀, or PEG₁₀₀₀ to PEG₁₀₀₀₀, or PEG₁₀₀₀ to PEG₅₀₀₀. In certain embodiments, the stealth agent can be PEG₅₀₀, PEG₁₀₀₀, PEG₂₀₀₀, or PEG₅₀₀₀. In certain embodiments, PEG can be terminated with an amine, methyl ether, an alcohol, or a carboxylic acid. In certain embodiments, the stealth agent can include at least two PEG molecules each linked together with a linking group. Linking groups can include those described above, e.g., amide linkages. In some embodiments, PEGylated-lipids are present in a bilayer of the nanoparticle, e.g., a liposome, in an amount sufficient to make the nanoparticle “stealth,” wherein a stealth nanoparticle shows reduced immunogenicity.

### Therapeutic Agents

The nanoparticles used in the targeted therapeutic or diagnostic delivery compositions of the present invention include a therapeutic agent, diagnostic agent, or a combination thereof. The therapeutic agent and/or diagnostic agent can be present anywhere in, on, or around the nanoparticle. In some embodiments, the therapeutic agent and/or diagnostic agent can be embedded in, encapsulated in, or tethered to the nanoparticle. In certain embodiments, the nanoparticle is a liposome and the diagnostic and/or therapeutic agent is encapsulated in the liposome.

A therapeutic agent used in the present invention can include any agent directed to treat a condition in a subject. In general, any therapeutic agent known in the art can be used, including without limitation agents listed in the United States Pharmacopeia (U.S.P.), Goodman and Gilman's The Pharmacological Basis of Therapeutics, 11th ed., McGraw Hill, 2005; Katzung, Ed., Basic and Clinical Pharmacology, McGraw-Hill/Appleton & Lange, 11th ed., Sep. 21, 2009; Physician's Desk Reference, PDR Network, 64th ed. 2010; The Merck Manual of Diagnosis and Therapy, Merck, 18th ed., 2006; or, in the case of animals, The Merck Veterinary Manual, 10th ed., Kahn Ed., Merck, 2010; all of which are incorporated herein by reference.

Therapeutic agents can be selected depending on the type of disease desired to be treated. For example, certain types of cancers or tumors, such as carcinoma, sarcoma, leukemia, lymphoma, myeloma, and central nervous system cancers as well as solid tumors and mixed tumors, can involve administration the same or possibly different therapeutic agents. In certain embodiments, a therapeutic agent can be delivered to treat or affect a cancerous condition in a subject and can include chemotherapeutic agents, such as alkylating agents, antimetabolites, anthracyclines, alkaloids, topoisomerase inhibitors, and other anticancer agents. In some embodiments, the agents can include antisense agents, microRNA, and/or siRNA agents.

In some embodiments, a therapeutic agent can include an anticancer agent or cytotoxic agent including but not limited to avastin, doxorubicin, cisplatin, oxaliplatin, carboplatin, 5-fluorouracil, gemcitibine or taxanes, such as paclitaxel and docetaxel. Additional anti-cancer agents can include but are not limited to 20-epi-1,25 dihydroxyvitamin D3,4-ipomeanol, 5-ethynyluracil, 9-dihydrotaxol, abiraterone, acivicin, aclarubicin, acodazole hydrochloride, acronine, acylfulvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizing morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisaziridinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caracemide, carbetimer, carboplatin, carboxamide-amino-triazole, carboxyamidotriazole, carest M3, carmustine, cam 700, cartilage derived inhibitor, carubicin hydrochloride, carzelesin, casein kinase inhibitors, castanospermine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethylnorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocarmycin SA, ebselen, ecomustine, edatrexate, edelfosine, edrecolomab, eflomithine, eflomithine hydrochloride, elemene, elsamitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, fluorocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ilmofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-IB, interferons, interleukins, iobenguane, iododoxorubicin, iproplatin, irinotecan, irinotecan hydrochloride, iroplact, irsogladine, isobengazole, isohomohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide 7, lobaplatin, lombricine, lometrexol, lometrexol sodium, lomustine, lonidamine, losoxantrone, losoxantrone hydrochloride, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C inhibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, 06-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazofurin, pyrazoloacridine, pyridoxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RH retinamide, RNAi, rogletimide, rohitukine, romurtide, roquinimex, rubiginone B 1, ruboxyl, safingol, safingol hydrochloride, saintopin, sarcnu, sarcophytol A, sargramostim, SDI 1 mimetics, semustine, senescence derived inhibitor 1, sense oligonucleotides, signal transduction inhibitors, signal transduction modulators, simtrazene, single chain antigen binding protein, sizofuran, sobuzoxane, sodium borocaptate, sodium phenylacetate, solverol, somatomedin binding protein, sonermin, sparfosate sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine, vinorelbine tartrate, vinrosidine sulfate, vinxaltine, vinzolidine sulfate, vitaxin, vorozole, zanoterone, zeniplatin, zilascorb, zinostatin, zinostatin stimalamer, or zorubicin hydrochloride.

In some embodiments, the therapeutic agents can be part of cocktail of agents that includes administering two or more therapeutic agents. For example, a liposome having both cisplatin and oxaliplatin can be administered. In addition, the therapeutic agents can be delivered before, after, or with immune stimulatory adjuvants, such as aluminum gel or salt adjuvants (e.g., alumimum phosphate or aluminum hydroxide), calcium phosphate, endotoxins, toll-like receptor adjuvants and the like.

Therapeutic agents of the present invention can also include radionuclides for use in therapeutic applications. For example, emitters of Auger electrons, such as ¹¹¹In, can be combined with a chelate, such as diethylenetriaminepentaacetic acid (DTPA) or 1,4,7,10-tetraazacyclododecane-1,4,7,10-tetraacetic acid (DOTA), and included in a targeted delivery composition, such as a liposome, to be used for treatment. Other suitable radionuclide and/or radionuclide-chelate combinations can include but are not limited to beta radionuclides (¹⁷⁷Lu, ¹⁵³Sm, ^{88/90}Y) with DOTA, ⁶⁴Cu-TETA, ^{188/186}Re(CO)₃-IDA; ^{188/186}Re(CO)triamines (cyclic or linear), ^{188/186}Re(CO)₃-Enpy2, and ^{188/186}Re(CO)₃-DTPA.

As described above, the therapeutic agents used in the present invention can be associated with the nanoparticle in a variety of ways, such as being embedded in, encapsulated in, or tethered to the nanoparticle. Loading of the therapeutic agents can be carried out through a variety of ways known in the art, as disclosed for example in the following references: de Villiers, M. M. et al., Eds., Nanotechnology in Drug Delivery, Springer (2009); Gregoriadis, G., Ed., Liposome Technology: Entrapment of drugs and other materials into liposomes, CRC Press (2006). In a group of embodiments, one or more therapeutic agents can be loaded into liposomes. Loading of liposomes can be carried out, for example, in an active or passive manner. For example, a therapeutic agent can be included during the self-assembly process of the liposomes in a solution, such that the therapeutic agent is encapsulated within the liposome. In certain embodiments, the therapeutic agent may also be embedded in the liposome bilayer or within multiple layers of multilamellar liposome. In alternative embodiments, the therapeutic agent can be actively loaded into liposomes. For example, the liposomes can be exposed to conditions, such as electroporation, in which the bilayer membrane is made permeable to a solution containing therapeutic agent thereby allowing for the therapeutic agent to enter into the internal volume of the liposomes.

### Diagnostic Agents

A diagnostic agent used in the present invention can include any diagnostic agent known in the art, as provided, for example, in the following references: Armstrong et al., Diagnostic Imaging, 5^{th} Ed., Blackwell Publishing (2004); Torchilin, V. P., Ed., Targeted Delivery of Imaging Agents, CRC Press (1995); Vallabhajosula, S., Molecular Imaging: Radiopharmaceuticals for PET and SPECT, Springer (2009). A diagnostic agent can be detected by a variety of ways, including as an agent providing and/or enhancing a detectable signal that includes, but is not limited to, gamma-emitting, radioactive, echogenic, optical, fluorescent, absorptive, magnetic or tomography signals. Techniques for imaging the diagnostic agent can include, but are not limited to, single photon emission computed tomography (SPECT), magnetic resonance imaging (MRI), optical imaging, positron emission tomography (PET), computed tomography (CT), x-ray imaging, gamma ray imaging, and the like.

In some embodiments, a diagnostic agent can include chelators that bind, e.g., to metal ions to be used for a variety of diagnostic imaging techniques. Exemplary chelators include but are not limited to ethylenediaminetetraacetic acid (EDTA), [4-(1,4,8,11-tetraazacyclotetradec-1-yl)methyl]benzoic acid (CPTA), Cyclohexanediaminetetraacetic acid (CDTA), ethylenebis(oxyethylenenitrilo)tetraacetic acid (EGTA), diethylenetriaminepentaacetic acid (DTPA), citric acid, hydroxyethyl ethylenediamine triacetic acid (HEDTA), iminodiacetic acid (IDA), triethylene tetraamine hexaacetic acid (TTHA), 1,4,7,10-tetraazacyclododecane-1,4,7,10-tetra(methylene phosphonic acid) (DOTP), 1,4,8,11-tetraazacyclododecane-1,4,8,11-tetraacetic acid (TETA), 1,4,7,10-tetraazacyclododecane-1,4,7,10-tetraacetic acid (DOTA), and derivatives thereof.

A radioisotope can be incorporated into some of the diagnostic agents described herein and can include radionuclides that emit gamma rays, positrons, beta and alpha particles, and X-rays. Suitable radionuclides include but are not limited to ²²⁵Ac, ⁷²As, ²¹¹At, ¹¹B, ¹²⁸Ba, ²¹²Bi, ⁷⁵Br, ⁷⁷Br, ¹⁴C, ¹⁰⁹Cd, ⁶²Cu, ⁶⁴Cu, ⁶⁷Cu, ¹⁸F, ⁶⁷Ga, ⁶⁸Ga, ³H, ¹²³I, ¹²⁵I, ¹³⁰I, ¹³¹I, ¹¹¹In, ¹⁷⁷Lu, ¹³N, ¹⁵O, ³²P, ³³P, ²¹²Pb, ¹⁰³Pd, ¹⁸⁶Re, ¹⁸⁸Re, ⁴⁷Sc, ¹⁵³Sm, ⁸⁹Sr, ^{99m}TC, ⁸⁸Y and ⁹⁰Y. In certain embodiments, radioactive agents can include ¹¹¹In-DTPA, ^{99m}Tc(CO)₃-DTPA, ^{99m}Tc(CO)₃-ENPy2, ^{62/64/67}Cu-TETA, ^{99m}Tc(CO)₃-IDA, and ^{99m}Tc(CO)₃-triamines (cyclic or linear). In other embodiments, the agents can include DOTA and its various analogs with ¹¹¹In, ¹⁷⁷Lu, ¹⁵³Sm, ^{88/90}Y, ^{62/64/67}Cu, or ^{67/68}Ga. In some embodiments, the liposomes can be radiolabeled, for example, by incorporation of lipids attached to chelates, such as DTPA-lipid, as provided in the following references: Phillips et al., Wiley Interdisciplinary Reviews: Nanomedicine and Nanobiotechnology, 1(1): 69-83 (2008); Torchilin, V. P. & Weissig, V., Eds. Liposomes 2nd Ed.: Oxford Univ. Press (2003); Elbayoumi, T. A. & Torchilin, V. P., Eur. J. Nucl. Med. Mol. Imaging. 33:1196-1205 (2006); Mougin-Degraef, M. et al., Int'l J. Pharmaceutics 344:110-117 (2007).

In other embodiments, the diagnostic agents can include optical agents such as fluorescent agents, phosphorescent agents, chemiluminescent agents, and the like. Numerous agents (e.g., dyes, probes, labels, or indicators) are known in the art and can be used in the present invention. (See, e.g., Invitrogen, The Handbook—A Guide to Fluorescent Probes and Labeling Technologies, Tenth Edition (2005)). Fluorescent agents can include a variety of organic and/or inorganic small molecules or a variety of fluorescent proteins and derivatives thereof. For example, fluorescent agents can include but are not limited to cyanines, phthalocyanines, porphyrins, indocyanines, rhodamines, phenoxazines, phenylxanthenes, phenothiazines, phenoselenazines, fluoresceins, benzoporphyrins, squaraines, dipyrrolo pyrimidones, tetracenes, quinolines, pyrazines, corrins, croconiums, acridones, phenanthridines, rhodamines, acridines, anthraquinones, chalcogenopyrylium analogues, chlorins, naphthalocyanines, methine dyes, indolenium dyes, azo compounds, azulenes, azaazulenes, triphenyl methane dyes, indoles, benzoindoles, indocarbocyanines, benzoindocarbocyanines, and BODIPY™ derivatives having the general structure of 4,4-difluoro-4-bora-3a,4a-diaza-s-indacene, and/or conjugates and/or derivatives of any of these. Other agents that can be used include, but are not limited to, for example, fluorescein, fluorescein-polyaspartic acid conjugates, fluorescein-polyglutamic acid conjugates, fluorescein-polyarginine conjugates, indocyanine green, indocyanine-dodecaaspartic acid conjugates, indocyanine (NIRD)-polyaspartic acid conjugates, isosulfan blue, indole disulfonates, benzoindole disulfonate, bis(ethylcarboxymethyl)indocyanine, bis(pentylcarboxymethyl)indocyanine, polyhydroxyindole sulfonates, polyhydroxybenzoindole sulfonate, rigid heteroatomic indole sulfonate, indocyaninebispropanoic acid, indocyaninebishexanoic acid, 3,6-dicyano-2,5-[(N,N,N′,N′-tetrakis(carboxymethyl)amino]pyrazine, 3,6-[(N,N,N′,N′-tetrakis(2-hydroxyethyl)amino]pyrazine-2,5-dicarboxylic acid, 3,6-bis(N-azatedino)pyrazine-2,5-dicarboxylic acid, 3,6-bis(N-morpholino)pyrazine-2,5-dicarboxylic acid, 3,6-bis(N-piperazino)pyrazine-2,5-dicarboxylic acid, 3,6-bis(N-thiomorpholino)pyrazine-2,5-dicarboxylic acid, 3,6-bis(N-thiomorpholino)pyrazine-2,5-dicarboxylic acid S-oxide, 2,5-dicyano-3,6-bis(N-thiomorpholino)pyrazine S,S-dioxide, indocarbocyaninetetrasulfonate, chloroindocarbocyanine, and 3,6-diaminopyrazine-2,5-dicarboxylic acid.

One of ordinary skill in the art will appreciate that particular optical agents used can depend on the wavelength used for excitation, depth underneath skin tissue, and other factors generally well known in the art. For example, optimal absorption or excitation maxima for the optical agents can vary depending on the agent employed, but in general, the optical agents of the present invention will absorb or be excited by light in the ultraviolet (UV), visible, or infrared (IR) range of the electromagnetic spectrum. For imaging, dyes that absorb and emit in the near-IR (˜700-900 nm, e.g., indocyanines) are preferred. For topical visualization using an endoscopic method, any dyes absorbing in the visible range are suitable.

In some embodiments, the non-ionizing radiation employed in the process of the present invention can range in wavelength from about 350 nm to about 1200 nm. In one exemplary embodiment, the fluorescent agent can be excited by light having a wavelength in the blue range of the visible portion of the electromagnetic spectrum (from about 430 nm to about 500 nm) and emits at a wavelength in the green range of the visible portion of the electromagnetic spectrum (from about 520 nm to about 565 nm). For example, fluorescein dyes can be excited with light with a wavelength of about 488 nm and have an emission wavelength of about 520 nm. As another example, 3,6-diaminopyrazine-2,5-dicarboxylic acid can be excited with light having a wavelength of about 470 nm and fluoresces at a wavelength of about 532 nm. In another embodiment, the excitation and emission wavelengths of the optical agent may fall in the near-infrared range of the electromagnetic spectrum. For example, indocyanine dyes, such as indocyanine green, can be excited with light with a wavelength of about 780 nm and have an emission wavelength of about 830 nm.

In yet other embodiments, the diagnostic agents can include but are not limited to contrast agents that are generally well known in the art, including, for example, superparamagnetic iron oxide (SPIO), complexes of gadolinium or manganese, and the like. (See, e.g., Armstrong et al., Diagnostic Imaging, 5^{th} Ed., Blackwell Publishing (2004)). In some embodiments, a diagnostic agent can include a magnetic resonance (MR) imaging agent. Exemplary magnetic resonance agents include but are not limited to paramagnetic agents, superparamagnetic agents, and the like. Exemplary paramagnetic agents can include but are not limited to Gadopentetic acid, Gadoteric acid, Gadodiamide, Gadolinium, Gadoteridol, Mangafodipir, Gadoversetamide, Ferric ammonium citrate, Gadobenic acid, Gadobutrol, or Gadoxetic acid. Superparamagnetic agents can include but are not limited to superparamagnetic iron oxide and Ferristene. In certain embodiments, the diagnostic agents can include x-ray contrast agents as provided, for example, in the following references: H. S Thomsen, R. N. Muller and R. F. Mattrey, Eds., Trends in Contrast Media, (Berlin: Springer-Verlag, 1999); P. Dawson, D. Cosgrove and R. Grainger, Eds., Textbook of Contrast Media (ISIS Medical Media 1999); Torchilin, V. P., Curr. Pharm. Biotech. 1:183-215 (2000); Bogdanov, A. A. et al., Adv. Drug Del. Rev. 37:279-293 (1999); Sachse, A. et al., Investigative Radiology 32(1):44-50 (1997). Examples of x-ray contrast agents include, without limitation, iopamidol, iomeprol, iohexyl, iopentol, iopromide, iosimide, ioversol, iotrolan, iotasul, iodixanol, iodecimol, ioglucamide, ioglunide, iogulamide, iosarcol, ioxilan, iopamiron, metrizamide, iobitridol and iosimenol. In certain embodiments, the x-ray contrast agents can include iopamidol, iomeprol, iopromide, iohexyl, iopentol, ioversol, iobitridol, iodixanol, iotrolan and iosimenol.

Similar to therapeutic agents described above, the diagnostic agents can be associated with the nanoparticle in a variety of ways, including for example being embedded in, encapsulated in, or tethered to the nanoparticle. Similarly, loading of the diagnostic agents can be carried out through a variety of ways known in the art, as disclosed for example in the following references: de Villiers, M. M. et al., Eds., Nanotechnology in Drug Delivery, Springer (2009); Gregoriadis, G., Ed., Liposome Technology: Entrapment of drugs and other materials into liposomes, CRC Press (2006).

### Preferential Binding Pairs

As provided herein, a preferential binding pair of the present invention includes a pair of molecules that bind to each other, e.g., oligonucleotides or oligonucleotide mimics, typically in a specific manner. In certain embodiments, a preferential binding pair can include one oligonucleotide member that has a preference for binding to a single or a plurality of DNA sequences over others, e.g., a second oligonucleotide member. For a given oligonucleotide, there are a spectrum of differential affinities for different DNA sequences ranging from non-sequence-specific (no detectable preference) to sequence preferential to absolute sequence specificity (i.e., the recognition of only a single sequence among all possible sequences). In the present invention, C¹ and C² are members of a preferential binding pair. In certain exemplary embodiments, C¹ can be one member of a preferential binding pair with a second member C², such that C¹ and C² can be oligonucleotides or oligonucleotide mimics. In certain embodiments, C¹ and C² can include nucleotide sequences that hybridize to one another but do not hybridize to any nucleotide sequence present in a subject. In some embodiments, C¹ and C² can be administered to a subject as part of a targeted delivery composition of the invention so that there is no competitive binding between C¹ and/or C² and another molecule present in the subject. In some embodiments, oligonucleotide sequences can be sequences that do not occur in nature, i.e., non-natural sequences.

Preferential binding members, such as C¹ and C², can include oligonucleotides or and/or oligonucleotide mimics that span a wide range of lengths. For example, oligonucleotides and/or oligonucleotide mimics can range in length from 2 to 100 units. In some embodiments, oligonucleotides can range from about 2 to about 100 nucleic acids in length, from about 2 to about 50 nucleic acids in length, from about 8 to about 50 nucleic acids in length, from about 8 to about 40 nucleic acids in length, from about 10 to about 30 nucleic acids in length, or from about 20 to about 30 nucleic acids in length.

Generally, C¹ and C² can respectively include oligonucleotides that form a duplex which is stable under conditions that are suitable for delivery and transport of the targeted delivery composition in a subject undergoing therapy or diagnosis. The preferential nature of a binding pair can be described in a variety of ways, such as by melting temperature or complementarity between the two binding pair members. It is well known that single strand oligonucleotides readily form duplex DNA upon contact with a single strand oligonucleotide having a complementary sequence. In some embodiments, C¹ and C² can respectively include complementary oligonucleotide sequences that can be greater than about 95% complementary, greater than about 90% complementary, greater than about 85% complementary, greater than about 80% complementary, greater than about 75% complementary, greater than about 70% complementary, greater than about 60% complementary, or greater than about 50% complementary. In certain embodiments, C¹ or C² may be longer than one another or the same length. C¹ can also be complementary along a portion of C² or vice versa. For example, C¹ can be at least 60% complementary, at least 70% complementary, at least 80% complementary, or at least 90% complementary to a portion of C² or vice versa. In one embodiment, C¹ and C² can be 40 nucleic acids in length and C¹ and C² are at least 70% complementary over a portion that is about 8 to about 30 nucleic acids in length. In yet another embodiment, C¹ and C² can include oligonucleotides having from 12-25 nucleic acids and being greater than 90% complementary.

Methods of predicting duplex DNA stability and melting temperatures between two sequences are well known (described in, e.g., Breslauer, K. J., et al., Proc. Natl. Acad. Sci. USA, 83, 3746-3750 (1986), Owczarzy R. et al., Biopolymers 44, 217-239 (1997); Sugimoto N. et al., Biochemistry 34, 11211-11216 (1995); Owczarzy R. et al., Biochemistry 43, 3537-3554 (2004)). Accordingly, sequences of C¹ and C² can be constructed in a way to make the two members preferentially bind to one another under certain conditions that in some instances can be pre-determined. In some embodiments, the preferential binding pairs used in the present invention encompass sequences that have melting temperatures above the body temperature of a subject being treated. In certain embodiments, the melting temperature can be greater than at least about 37° C., greater than at least about 38° C., greater than at least about 39° C., greater than at least about 40° C., or greater than at least about 41° C. In other embodiments, the melting temperature of a preferential binding pair can range between about 37° C. and about 41° C., between about 40° C. and 50° C., or between about 40° C. and about 60° C. In yet other embodiments, the preferential binding pair can be pre-designed to have a melting temperature at least 1° C., at least 2° C., at least 3° C., at least 4° C., at least 5° C., at least 10° C., or at least 20° C. above the body temperature of a subject. One of ordinary skill in the art will appreciate that particular sequences can be predicted to have certain melting temperatures, specific for a particular use of the present invention.

Members of a preferential binding pair can also include oligonucleotide mimics capable of preferentially binding to one another. In some embodiments, the oligonucleotide mimics can form duplexes together (e.g., PNA/PNA) or with oligonucleotides (e.g., PNA/DNA or PNA/RNA). In certain embodiments, the oligonucleotide mimics and/or oligonucleotides can hybridize via interactions other than Watson-Crick hydrogen bonding rules, and can form stable duplexes in solution. (See, e.g., Egholm et al., Nature 365: 566-568 (1993)).

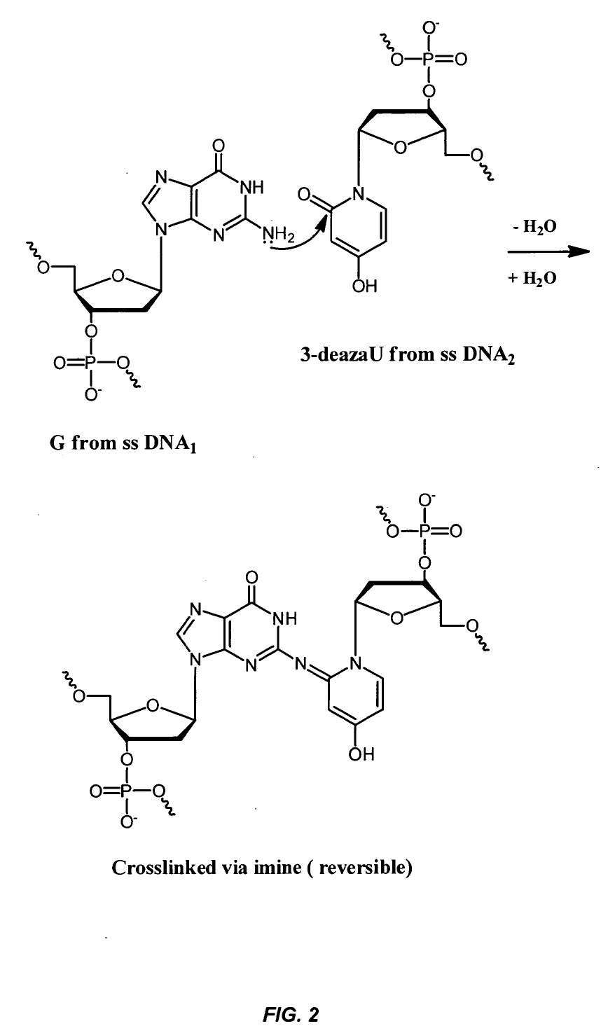

In yet another embodiment, targeted delivery compositions can be modified to be more robust. For example, after members of a preferential binding pair, such as C¹ and C², have hybridized, the two complementary strands of DNA, RNA, and/or PNA can be further cross-linked by a variety of methods known in the art. (See, e.g., Webb, Thomas R., Matteucci, Mark D., Nucleic Acids Research (1986) 14(19), 7661-7674). One of ordinary skill in the art will appreciate that a variety of crosslinking agents can be used and that a variety of chemistries, e.g., photocrosslinking or chemical crosslinking, can be employed. In addition, a variety of linking moieties can be attached to the oligonucleotides in several ways, such as covalent attachment to an oligonucleotide and/or during synthesis of the oligonucleotides. In certain embodiments, the stability of the duplex can be increased by incorporating at least one linking moiety capable of forming a covalent crosslink between oligonucleotide strands. As shown for example in FIG. 2, 3—deoxyuridine in one of the oligonucleotide strands (e.g., a member of a preferential binding pair, such as C¹) can be situated in the sequence so that it can hybridize across from a guanine (G) present in a complementary oligonucleotide sequence (the other member of a preferential binding pair, such as C²). Crosslinking thereby results in formation of a covalently crosslinked duplex pair.

### Targeting Components

The targeted delivery compositions of the present invention also include a targeting component having the formula: C²-(L²)_{y}-T. The linking group, L², and the member of a preferential binding pair, C², are described in more detail above. The subscript y is generally 0 or 1.

The targeted delivery compositions of the present invention also include T, a targeting agent. Generally, the targeting agents of the present invention can associate with any target of interest, such as a target associated with an organ, tissues, cell, extracellular matrix, or intracellular region. In certain embodiments, a target can be associated with a particular disease state, such as a cancerous condition. Alternatively, a targeting component can target one or more particular types of cells that can, for example, have a target that indicates a particular disease and/or particular state of a cell, tissue, and/or subject. In some embodiments, the targeting component can be specific to only one target, such as a receptor. Suitable targets can include but are not limited to a nucleic acid, such as a DNA, RNA, or modified derivatives thereof. Suitable targets can also include but are not limited to a protein, such as an extracellular protein, a receptor, a cell surface receptor, a tumor-marker, a transmembrane protein, an enzyme, or an antibody. Suitable targets can include a carbohydrate, such as a monosaccharide, disaccharide, or polysaccharide that can be, for example, present on the surface of a cell. In certain embodiments, suitable targets can include mucins such as MUC-1 and MUC-4, growth factor receptors such as EGFR, Claudin 4, nucleolar phosphoproteins such as nucleolin, chemokine receptors such as CCR7, receptors such as somatostatin receptor 4, Erb-B2 (erythroblastic leukaemia oncogene homologue 2) receptor, CD44 receptor, and VEGF receptor-2 kinase.

In certain embodiments, a targeting agent can include a small molecule mimic of a target ligand (e.g., a peptide mimetic ligand), a target ligand (e.g., an RGD peptide containing peptide or folate amide), or an antibody or antibody fragment specific for a particular target. In some embodiments, a targeting agent can further include folic acid derivatives, B-12 derivatives, integrin RGD peptides, NGR derivatives, somatostatin derivatives or peptides that bind to the somatostatin receptor, e.g., octreotide and octreotate, and the like.

The targeting agents of the present invention can also include an aptamer. Aptamers can be designed to associate with or bind to a target of interest. Aptamers can be comprised of, for example, DNA, RNA, and/or peptides, and certain aspects of aptamers are well known in the art. (See. e.g., Klussman, S., Ed., The Aptamer Handbook, Wiley-VCH (2006); Nissenbaum, E. T., Trends in Biotech. 26(8): 442-449 (2008)). In the present invention, suitable aptamers can be linear or cyclized and can include oligonucleotides having less than about 150 bases (i.e., less than about 150 mer). Aptamers can range in length from about 100 to about 150 bases or from about 80 to about 120 bases. In certain embodiments, the aptamers can range from about 12 to 40 about bases, from about 12 to about 25 bases, from about 18 to about 30 bases, or from about 15 to about 50 bases. The aptamers can be developed for use with a suitable target that is present or is expressed at the disease state, and includes, but is not limited to, the target sites noted herein.

B. Targeted Delivery Compositions Including a Diagnostic and/or Therapeutic Agent Directly Attached to a Linking Group

In another aspect, the present invention provides targeted delivery compositions wherein a diagnostic and/or therapeutic agent is directly attached to a linking group. In one embodiment, the targeted delivery compositions of the present invention include a targeted delivery composition, comprising: (a) a diagnostic or therapeutic component having the formula: DT-(L¹)ₓ-C¹; (b) a targeting component having the formula: C²-(L²)_{y}-T, wherein, DT is a therapeutic agent, diagnostic agent, or a combination thereof; each of L¹ and L² is a hydrophilic, non-immunogenic, water soluble linking group; C¹ is one member of a preferential binding pair with a second member C², wherein C¹ and C² are oligonucleotides or oligonucleotide mimics; T is a targeting agent; and each of the subscripts x and y are independently 0 or 1, but at least one of x and y is other than 0.

In another embodiment, the present invention provides a targeted therapeutic or diagnostic delivery composition, comprising: (a) a nanoparticle; (b) a derivatized attachment component having the formula: A-(L¹)ₓ-C¹; and (c) a diagnostic or therapeutic component having the formula: C²-(L²)_{y}-DT wherein, A is an attachment component; each of L¹ and L² is a hydrophilic, non-immunogenic, water soluble linking group; C¹ is one member of a preferential binding pair with a second member C², wherein C¹ and C² are oligonucleotides or oligonucleotide mimics; DT is a therapeutic agent, diagnostic agent, or a combination thereof; and each of the subscripts x and y are independently 0 or 1, but at least one of x and y is other than 0; wherein the A portion of said derivatized attachment component is attached to the nanoparticle.

In general, it will be appreciated by one of ordinary skill in the art that the selected embodiments of the targeted delivery compositions including a nanoparticle as described above can be similarly applied to the embodiments disclosed herein for targeted delivery compositions wherein a diagnostic and/or therapeutic agent is directly attached to a linking group. Methods for attaching the diagnostic and/or therapeutic agents to the linking groups are well known in the art, and are typically covalent attachments that are described in more detail above. It will be appreciated by one of ordinary skill in the art that functional groups and/or bifunctional linkers (each described in detail above) can be used to attach, for example, DT to a linking group (L¹ or L²). In addition, DT can include any of the therapeutic and/or diagnostic agents that are described above and directly provides the therapeutic and/or diagnostic agent to a subject without the need for a nanoparticle. Similarly, the targeting components can be the same as the targeting components used for nanoparticle-based targeted delivery compositions, as described above. Also, members of a preferential binding pair, such as C¹ and C², are the same as those described above in relation to targeted delivery compositions including a nanoparticle.

### C. Individual Components of the Targeted Delivery Compositions

In yet another aspect, the present invention provides individual components of the targeted delivery compositions disclosed herein. In particular, the present invention includes a derivatized attachment component having the formula: A-(L¹)-C¹, wherein, A is an attachment component; L¹ is a hydrophilic, non-immunogenic, water soluble linking group; and C¹ is one member of a preferential binding pair with a second member C², wherein C¹ and C² are oligonucleotides or oligonucleotide mimics.

In yet another aspect, the present invention includes a targeting component having the formula: C²-(L²)-T wherein, L² is a hydrophilic, non-immunogenic, water soluble linking group; C² is one member of a preferential binding pair with a second member C¹, wherein C¹ and C² are oligonucleotides or oligonucleotide mimics; and T is a targeting agent.

In yet another aspect, the present invention includes a diagnostic or therapeutic component having the formula: (DT)-(L¹)-C¹ wherein, DT is a therapeutic agent, diagnostic agent, or a combination thereof; L¹ is a hydrophilic, non-immunogenic, water soluble linking group; and C¹ is one member of a preferential binding pair with a second member C², wherein C¹ and C² are oligonucleotides or oligonucleotide mimics.

It will be appreciated by one of ordinary skill in the art that each of the components of the targeted delivery compositions similarly include each of the specific embodiments described above.

### IV. Methods of Preparing Targeted Delivery Compositions and Components

### A. Targeted Delivery Compositions Including a Nanoparticle

The targeted delivery compositions of the present invention can be produced in a variety of ways. In one aspect, targeted delivery compositions of the present invention can be prepared using a method of preparing a targeted therapeutic or diagnostic delivery composition, comprising contacting a derivatized attachment component having the formula: A-(L¹)ₓ-C¹; with a targeting component having the formula: C²-(L²)_{y}-T wherein, A is an attachment component for attaching the derivatized attachment component to the nanoparticle; each of L¹ and L² is a hydrophilic, non-immunogenic, water soluble linking group; C¹ is one member of a preferential binding pair with a second member C², wherein C¹ and C² are oligonucleotides or oligonucleotide mimics; T is a targeting agent; and each of the subscripts x and y are independently 0 or 1, but at least one of x and y is other than 0; wherein the A portion of said derivatized attachment component is attached to a nanoparticle under conditions sufficient to attach A to the nanoparticle; and the nanoparticle-A-(L¹)ₓ-C¹ conjugate is subsequently contacted with the targeting component under conditions sufficient for a duplex to be formed between C¹ and C².

In general, the targeted delivery compositions of the invention can be assembled in one step or in a step-wise fashion that can be conducted in any order. For example, a derivatized attachment component can be attached to a nanoparticle including a therapeutic and/or diagnostic agent. The targeting component can then be added to the targeted delivery composition by hybridization between the members of the preferential binding pair. In an alternative embodiment, all of the components (e.g., the nanoparticles, the derivatized attachment components, and the targeting components) can be combined together to self-assemble together in a solution. In certain embodiments, the nanoparticle can include a liposome, and the derivatized attachment component can be included during formation of the liposomes. The targeting component can be added after liposome formation with the derivatized attachment component. Alternatively, the targeting component can be included during the self-assembly process of the liposomes, so as to form a complete targeted delivery composition after self-assembly.

### Nanoparticles

Nanoparticles can be produced by a variety of ways generally known in the art and methods of making such nanoparticles can depend on the particular nanoparticle desired. Any measuring technique available in the art can be used to determine properties of the targeted delivery compositions and nanoparticles. For example, techniques such as dynamic light scattering, x-ray photoelectron microscopy, powder x-ray diffraction, scanning electron microscopy (SEM), transmission electron microscopy (TEM), and atomic force microscopy (AFM) can be used to determine average size and dispersity of the nanoparticles and/or targeted delivery compositions.

Liposomes used in the targeted delivery compositions of the present invention can be made using a variety of techniques generally well known in the art. (See, e.g., Williams, A. P., Liposomes: A Practical Approach, 2n^{d} Edition, Oxford Univ. Press (2003); Lasic, D. D., Liposomes in Gene Delivery, CRC Press LLC (1997)). For example, liposomes can be produced by but are not limited to techniques such as extrusion, agitation, sonication, reverse phase evaporation, self-assembly in aqueous solution, electrode-based formation techniques, microfluidic directed formation techniques, and the like. In certain embodiments, methods can be used to produce liposomes that are multilamellar and/or unilamellar, which can include large unilamellar vesicles (LUV) and/or small unilamellar vesicles (SUV). Similar to self-assembly of liposomes in solution, micelles can be produced using techniques generally well known in the art, such that amphiphilic molecules will form micelles when dissolved in solution conditions sufficient to form micelles. Lipid-coated bubbles and lipoproteins can also be constructed using methods known in the art (See, e.g., Farook, U., J. R. Soc. Interface, 6(32): 271-277 (2009); Lacko et al., Lipoprotein Nanoparticles as Delivery Vehicles for Anti-Cancer Agents in Nanotechnology for Cancer Therapy, CRC Press (2007)).

Methods of making polymeric nanoparticles that can be used in the present invention are generally well known in the art (See, e.g., Sigmund, W. et al., Eds., Particulate Systems in Nano- and Biotechnologies, CRC Press LLC (2009); Karnik et al., Nano Lett., 8(9): 2906-2912 (2008)). For example, block copolymers can be made using synthetic methods known in the art such that the block copolymers can self-assemble in a solution to form polymersomes and/or block copolymer micelles. Niosomes are known in the art and can be made using a variety of techniques and compositions (Baillie A. J. et al., J. Pharm. Pharmacol., 38:502-505 (1988)). Magnetic and/or metallic particles can be constructed using any method known in the art, such as co-precipitation, thermal decomposition, and microemulsion. (See also Nagarajan, R. & Hatton, T. A., Eds., Nanoparticles Synthesis, Stabilization, Passivation, and Functionalization, Oxford Univ. Press (2008)). Quantum dots or semiconductor nanocrystals can be synthesized using any method known in the art, such as colloidal synthesis techniques. Generally, quantum dots can be composed of a variety of materials, such as semiconductor materials including cadmium selenide, cadmium sulfide, indium arsenide, indium phosphide, and the like.

### Derivatized Attachment Components

The derivatized attachment component of the present invention can be manufactured using generally known methods in the art of chemical synthesis. For example, the oligonucleotide and/or oligonucleotide mimic portion (e.g., C¹) of the derivatized attachment component can be produced in a separate reaction synthesis from other portions of the derivatized attachment component. Oligonucleotide synthesis can be performed using a variety of methods known in the art and can depend on the length of the oligonucleotide. For shorter oligonucleotides, e.g., 20 to 30 nucleotides, phosphoramidite synthesis can be used. For longer oligonucleotides, e.g., 5000 nucleotides, conventional cloning techniques can be used to make the oligonucleotides, as described, e.g., in Smith et al., PNAS, 100(26): 15440-15445 (2003). Methods generally well known in the art can be used to isolate the nucleotide products. Subsequently, the synthesized oligonucleotide and/or oligonucleotide mimic (e.g., C¹) can be covalently attached at the 3′ or 5′ end to the hydrophilic, non-immunogenic, water soluble linking group using a variety of linking chemistries known in the art, as described herein. In certain embodiments, the oligonucleotide, e.g., C¹, is attached to the terminus of a hydrophilic, non-immunogenic, water soluble linking group. In an alternative aspect, the A portion, or the attachment component, can be attached to the hydrophilic, non-immunogenic, water soluble linking group (e.g., L¹) and then the oligonucleotide or oligonucleotide mimic can be synthesized onto the end of the linking group opposite the attachment component.

In one aspect, the hydrophilic, non-immunogenic, water soluble linking group can be attached to a phospholipid, such as distearoylphosphoethanolamine, using conventional chemistry known in the art. The terminus of a member of the preferential binding pair (e.g., the 3′ or 5′ end of an oligonucleotide represent C¹) can then be attached to the other end of the polyethylene glycol group using the techniques described above. In other embodiments, the member of the preferential binding pair, C¹, can be attached directly to the attachment component without a hydrophilic, non-immunogenic, water soluble linking group.

### Targeting Components

The targeting components of the present invention can be constructed using similar methods as disclosed above for the derivatized attachment components. The member of a preferential binding pair (e.g., C²) can be synthesized separately using oligonucleotide synthesis techniques described above. The member of the preferential binding pair can then be attached to one end of the hydrophilic, non-immunogenic, water soluble linking group (e.g., L²). Subsequently or prior to attachment of the preferential binding pair member, a targeting agent can be attached to the opposite end of the hydrophilic, non-immunogenic, water soluble linking group. In certain embodiments, the hydrophilic, non-immunogenic, water soluble linking group can be synthesized using the methods generally known in the art, prior to attachment to the member of a preferential binding pair or the targeting agent.

As will be appreciated by one of ordinary skill in the art, targeting agents of the present invention can be attached to the hydrophilic, non-immunogenic, water soluble linking group by a variety of ways that can depend on the characteristics of the targeting agent. For example, reaction syntheses can be different if the targeting agent is composed of peptides, nucleotides, carbohydrates, and the like.

In certain embodiments, the targeting agent can include an aptamer. Aptamers for a particular target can be identified using techniques known in the art, such as but not limited to, in vitro selection processes, such as SELEX (systematic evolution of ligands by exponential enrichment), or MonoLex™ technology (single round aptamer isolation procedure for AptaRes AG), in vivo selection processes, or combinations thereof (See e.g., Ellington, A. D. & Szostak, J. W., Nature 346(6287): 818-22; Bock et al., Nature 355(6360): 564-6 (1992)). In some embodiments, the above mentioned methods can be used to indentify particular DNA or RNA sequences that can be used to bind a particular target site of interest, as disclosed herein. Once a sequence of a particular aptamer has been identified, the aptamer can be constructed in a variety of ways known in the art, such as phosphoramidite synthesis. For peptide aptamers, a variety of identification and manufacturing techniques can be used (See e.g., Colas, P., J. Biol. 7:2 (2008); Woodman, R. et al., J. Mol. Biol. 352(5): 1118-33 (2005). Similar to reaction sequence described above regarding attachment of a member of the preferential binding pair, the hydrophilic, non-immunogenic, water soluble linking group of the targeting component can be reacted with a 3′ or 5′ end of the aptamer. In some embodiments, the aptamer can be attached to hydrophilic, non-immunogenic, water soluble linking group after the member of the preferential binding pair (e.g., C²) has been reacted with the other end of the hydrophilic, non-immunogenic, water soluble linking group. In other embodiments, the aptamer can be attached first and then followed by attachment of the preferentially binding pair member to form the targeting component. In alternative embodiments, the aptamer can be synthesized sequentially by adding one nucleic acid at a time to the end of the hydrophilic, non-immunogenic, water soluble linking group of the targeting component. In yet other embodiments, the preferential binding pair member and the targeting agent, e.g., the aptamer, can be placed in the same reaction vessel to form the targeting component all in one step.

B. Targeted Delivery Compositions Including a Diagnostic and/or Therapeutic Agent Directly Attached to a Linking Group

The targeted delivery compositions including a diagnostic and/or therapeutic agent directly attached to a linking group can be produced by several ways. In one aspect, the targeted delivery compositions can be produced using a method of preparing a targeted delivery composition, comprising contacting a diagnostic or therapeutic component having the formula: DT-(L¹)ₓ-C¹; with a targeting component having the formula: C²-(L²)_{y}-T wherein, DT is a therapeutic agent, diagnostic agent, or a combination thereof; each of L¹ and L² is a hydrophilic, non-immunogenic, water soluble linking group; C¹ is one member of a preferential binding pair with a second member C², wherein C¹ and C² are oligonucleotides or oligonucleotide mimics; T is a targeting agent; and each of the subscripts x and y are independently 0 or 1, but at least one of x and y is other than 0; under conditions sufficient for a duplex to be formed between C¹ and C².

In another aspect, targeted delivery compositions of the present invention can be prepared using a method of preparing a targeted therapeutic or diagnostic delivery composition, comprising contacting a derivatized attachment component having the formula: A-(L¹)ₓ-C¹; with a diagnostic or therapeutic component having the formula: C²-(L²)_{y}-DT wherein, A is an attachment component; each of L¹ and L² is a hydrophilic, non-immunogenic, water soluble linking group; C¹ is one member of a preferential binding pair with a second member C², wherein C¹ and C² are oligonucleotides or oligonucleotide mimics; DT is a therapeutic agent, diagnostic agent, or a combination thereof; and each of the subscripts x and y are independently 0 or 1, but at least one of x and y is other than 0; wherein the A portion of said derivatized attachment component is attached to said nanoparticle under conditions sufficient to attach A to the nanoparticle; and the nanoparticle-A-(L¹)ₓ-C¹ conjugate is subsequently contacted with the diagnostic or therapeutic component under conditions sufficient for a duplex to be formed between C¹ and C². It will be appreciated that other sequences of steps can be used to prepare targeted delivery compositions that include a diagnostic and/or therapeutic agent directly attached to a linking group.

### Diagnostic or Therapeutic Components

The diagnostic or therapeutic components having the formula DT-(L¹)ₓ-(C¹) can be prepared using methods generally well known in the art. In certain embodiments, a chelator can be attached to a hydrophilic, non-immunogenic, water soluble linking group and then a targeting agent can be attached to the other end of the linking group. A radioisotope can then be complexed with the chelator. The present invention, however, contemplates several orders of steps for making the conjugates. In some embodiments, certain steps can be reversed. For example, a chelator can be combined with a radioisotope to form the diagnostic component that can then be further reacted using conventional chemistry with a hydrophilic, non-immunogenic, water soluble linking group. The member of a preferential binding pair (C¹) can then be attached to the hydrophilic, non-immunogenic, water soluble linking group as described herein. In yet another aspect, a therapeutic agent can be attached to a hydrophilic, non-immunogenic, water soluble linking group and the member of a preferential binding pair (C¹) can be attached to the opposite end of the linking group, as described herein. One of ordinary skill in the art will appreciate that the diagnostic and/or therapeutic components can be constructed in several different ways other than the examples provided above. In addition, making the diagnostic or therapeutic components can depend on the particular diagnostic and/or therapeutic agent being used.

### IV. Methods of Administering Targeted Delivery Compositions

As described herein, the targeted delivery compositions and methods of the present invention can be used for treating and/or diagnosing any disease, disorder, and/or condition associated with a subject. In one embodiment, the methods of the present invention include a method for treating or diagnosing a cancerous condition in a subject, comprising administering to the subject a targeted delivery composition of the present invention that includes a nanoparticle, wherein the therapeutic or diagnostic agent is sufficient to treat or diagnose the condition. In certain embodiments, the cancerous condition can include cancers that sufficiently express (e.g., on the cell surface or in the vasculature) a receptor that is being targeted by a targeting agent of a targeted delivery composition of the present invention.

In another embodiment, the methods of the present invention include a method of determining the suitability of a subject for a targeted therapeutic treatment, comprising administering to said subject a targeted delivery composition that includes a nanoparticle, wherein the nanoparticle comprises a diagnostic agent, and imaging the subject to detect said diagnostic agent.

In yet another embodiment, the methods of the present invention include a method for treating or diagnosing a cancerous condition in a subject, comprising administering to the subject a targeted delivery composition of the present invention including a diagnostic and/or therapeutic agent directly attached to a linking group, wherein the therapeutic or diagnostic agent is sufficient to treat or diagnose the condition.

In yet another embodiment, the methods of the present invention include a method of determining the suitability of a subject for a targeted therapeutic treatment, comprising administering to said subject a targeted delivery composition of the present invention comprising a diagnostic agent directly attached to a linking group, and imaging said subject to detect said diagnostic agent.

### Administration

In some embodiments, the present invention can include a targeted delivery composition and a physiologically (i.e., pharmaceutically) acceptable carrier. As used herein, the term “carrier” refers to a typically inert substance used as a diluent or vehicle for a drug such as a therapeutic agent. The term also encompasses a typically inert substance that imparts cohesive qualities to the composition. Typically, the physiologically acceptable carriers are present in liquid form. Examples of liquid carriers include physiological saline, phosphate buffer, normal buffered saline (135-150 mM NaCl), water, buffered water, 0.4% saline, 0.3% glycine, glycoproteins to provide enhanced stability (e.g., albumin, lipoprotein, globulin, etc.), and the like. Since physiologically acceptable carriers are determined in part by the particular composition being administered as well as by the particular method used to administer the composition, there are a wide variety of suitable formulations of pharmaceutical compositions of the present invention (See, e.g., Remington's Pharmaceutical Sciences, 17^{th} ed., 1989).

The compositions of the present invention may be sterilized by conventional, well-known sterilization techniques or may be produced under sterile conditions. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents, wetting agents, and the like, e.g., sodium acetate, sodium lactate, sodium chloride, potassium chloride, calcium chloride, sorbitan monolaurate, and triethanolamine oleate. Sugars can also be included for stabilizing the compositions, such as a stabilizer for lyophilized targeted delivery compositions.

The targeted delivery composition of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which includes an effective amount of a packaged targeted delivery composition with a suppository base. Suitable suppository bases include natural or synthetic triglycerides or paraffin hydrocarbons. In addition, it is also possible to use gelatin rectal capsules which contain a combination of the targeted delivery composition of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can also be prepared from sterile powders, granules, and tablets. In the practice of the present invention, compositions can be administered, for example, by intravenous infusion, topically, intraperitoneally, intravesically, or intrathecally. Parenteral administration and intravenous administration are the preferred methods of administration. The formulations of targeted delivery compositions can be presented in unit-dose or multi-dose sealed containers, such as ampoules and vials.

The pharmaceutical preparation is preferably in unit dosage form. In such form the preparation is subdivided into unit doses containing appropriate quantities of the active component, e.g., a targeted delivery composition. The unit dosage form can be a packaged preparation, the package containing discrete quantities of preparation. The composition can, if desired, also contain other compatible therapeutic agents.

In therapeutic use for the treatment of cancer, the targeted delivery compositions including a therapeutic and/or diagnostic agent utilized in the pharmaceutical compositions of the present invention can be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the targeted delivery composition being employed. For example, dosages can be empirically determined considering the type and stage of cancer diagnosed in a particular patient. The dose administered to a patient, in the context of the present invention, should be sufficient to affect a beneficial therapeutic response in the patient over time. The size of the dose will also be determined by the existence, nature, and extent of any adverse side-effects that accompany the administration of a particular targeted delivery composition in a particular patient. Determination of the proper dosage for a particular situation is within the skill of the practitioner. Generally, treatment is initiated with smaller dosages which are less than the optimum dose of the targeted delivery composition. Thereafter, the dosage is increased by small increments until the optimum effect under circumstances is reached. For convenience, the total daily dosage may be divided and administered in portions during the day, if desired.

In some embodiments, the targeted delivery compositions of the present invention may be used to diagnose a disease, disorder, and/or condition. In some embodiments, the targeted delivery compositions can be used to diagnose a cancerous condition in a subject, such as lung cancer, breast cancer, pancreatic cancer, prostate cancer, cervical cancer, ovarian cancer, colon cancer, liver cancer, esophageal cancer, and the like. In some embodiments, methods of diagnosing a disease state may involve the use of the targeted delivery compositions to physically detect and/or locate a tumor within the body of a subject. For example, tumors can be related to cancers that sufficiently express (e.g., on the cell surface or in the vasculature) a receptor that is being targeted by a targeting agent of a targeted delivery composition of the present invention. In some embodiments, the targeted delivery compositions can also be used to diagnose diseases other than cancer, such as proliferative diseases, cardiovascular diseases, gastrointestinal diseases, genitourinary disease, neurological diseases, musculoskeletal diseases, hematological diseases, inflammatory diseases, autoimmune diseases, rheumatoid arthritis and the like.

As disclosed herein, the targeted delivery compositions of the invention can include a diagnostic agent that has intrinsically detectable properties. In detecting the diagnostic agent in a subject, the targeted delivery compositions, or a population of particles with a portion being targeted delivery compositions, can be administered to a subject. The subject can then be imaged using a technique for imaging the diagnostic agent, such as single photon emission computed tomography (SPECT), magnetic resonance imaging (MRI), optical imaging, positron emission tomography (PET), computed tomography (CT), x-ray imaging, gamma ray imaging, and the like. Any of the imaging techniques described herein may be used in combination with other imaging techniques. In some embodiments, the incorporation of a radioisotope for imaging in a particle allows in vivo tracking of the targeted delivery compositions in a subject. For example, the biodistribution and/or elimination of the targeted delivery compositions can be measured and optionally be used to alter the treatment of patient. For example, more or less of the targeted delivery compositions may be needed to optimize treatment and/or diagnosis of the patient.

### Targeted Delivery

In certain embodiments, the targeted delivery compositions of the present invention can be delivered to a subject to release a therapeutic or diagnostic agent in a targeted manner. For example, a targeted delivery composition can be delivered to a target in a subject and then a therapeutic agent embedded in, encapsulated in, or tethered to the targeted delivery composition, such as to the nanoparticle, can be delivered based on solution conditions in vicinity of the target. Solution conditions, such as pH, salt concentration, and the like, may trigger release over a short or long period of time of the therapeutic agent to the area in the vicinity of the target. Alternatively, an enzyme can cleave the therapeutic or diagnostic agent from the targeted delivery composition to initiate release. In some embodiments, the targeted delivery compositions can be delivered to the internal regions of a cell by endocytosis and possibly later degraded in an internal compartment of the cell, such as a lysosome. One of ordinary skill will appreciate that targeted delivery of a therapeutic or diagnostic agent can be carried out using a variety of methods generally known in the art.

### Kits

The present invention also provides kits for administering the targeted delivery compositions to a subject for treating and/or diagnosing a disease state. Such kits typically include two or more components necessary for treating and/or diagnosing the disease state, such as a cancerous condition. Components can include targeted delivery compositions of the present invention, reagents, containers and/or equipment. In some embodiments, a container within a kit may contain a targeted delivery composition including a radiopharmaceutical that is radiolabeled before use. The kits can further include any of the reaction components or buffers necessary for administering the targeted delivery compositions. Moreover, the targeted delivery compositions can be in lyophilized form and then reconstituted prior to administration.

In certain embodiments, the kits of the present invention can include packaging assemblies that can include one or more components used for treating and/or diagnosing the disease state of a patient. For example, a packaging assembly may include a container that houses at least one of the targeted delivery compositions as described herein. A separate container may include other excipients or agents that can be mixed with the targeted delivery compositions prior to administration to a patient. In some embodiments, a physician may be able to mix and match certain components and/or packaging assemblies depending on the treatment or diagnosis needed for a particular patient.

It is understood that the embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

The following examples describe example embodiments of how to make a derivatized attachment component, a diagnostic component, and targeting components, as described herein. In the examples, an attachment component includes a lipid coupled to a first oligonucleotide via a hydrophilic, non-immunogenic, water soluble linking group. In addition, a diagnostic component is provided in the form of a fluorescent agent coupled via a linking group to a second oligonucleotide that is complementary to the first oligonucleotide. Targeting components include a peptide targeting agent linked to an oligonucleotide as well as an aptamer targeting agent linked to an oligonucleotide. In certain examples, the derivatized attachment component can be incorporated into liposomes and then bound to a diagnostic component or targeting components via hybridization between preferential binding pairs. One of ordinary skill in the art will appreciate that the methods described in the examples can similarly to other derivatized attachment components, targeting components, and diagnostic or therapeutic components, as described herein.

### Example 1

### Preparation of 5′-DSPE-PEG (3400)-S—C₆H₁₂—VEGF Oligonucleotide Analog 1

5′-DSPE-PEG (3400)-S—C₆H₁₂—VEGF Oligonucleotide Analog 1 was prepared by the following steps:

### Step 1: Preparation of VEGF Oligonucleotide Analog 1

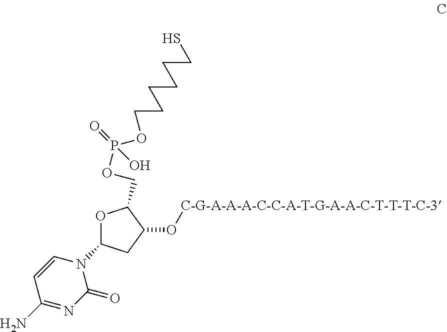

VEGF Oligonucleotide Analog 1, shown directly above, was prepared from commercially available protected nucleosides and an appropriately protected 6-hydroxyhexanethiol analog using commonly available solid support oligonucleotide synthesis techniques. Subsequent cleavage from the support and reverse phase purification gave 5′-VEGF Oligonucleotide Analog 1 as the 3′-free thiol in substantially pure form.

### Step 2: Preparation of 5′-DSPE-PEG (3400)-S—C₆H₁₂—VEGF Oligonucleotide Analog 1

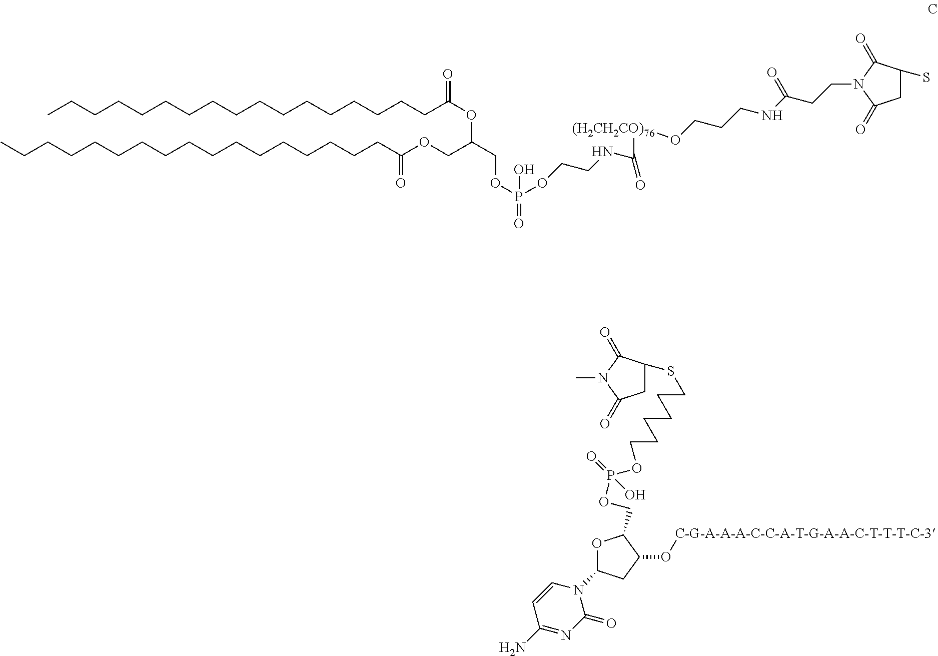

To produce 5′-DSPE-PEG (3400)-S—C₆H₁₂—VEGF Oligonucleotide Analog 1 (shown directly above), the product of Step 1 was reacted with DSPE-PEG 3400-maleimide in a suitable solvent. After reverse phase chromatography using a suitable water:acetonitrile gradient the title compound VEGF Oligonucleotide Analog 1-3-(3-(5-hydroxypentlthio)-2,5-dioxopyrrolidin-1-yl)propanamido-PEG 3400-DSPE conjugate was isolated in substantially pure form.

### Example 2

### Preparation of 5′-(6-FAM)-VEGF Oligonucleotide Analog 2

5′-(6-FAM (Fluorescein Amidite))—VEGF Oligonucleotide Analog 2 was prepared by the following steps:

### Step 1: Preparation of VEGF Oligonucleotide Analog 2

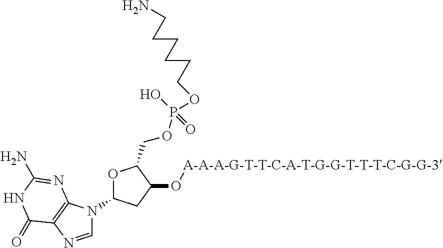

VEGF Oligonucleotide Analog 2, shown directly above, was prepared from commercially available protected nucleosides and 6-aminohexanol using commonly available solid support oligonucleotide synthesis techniques. Subsequent cleavage from the support and reverse phase purification gave VEGF oligonucleotide analog 2 in substantially pure form.

### Step 2: Preparation of 5′-(6-FAM)-VEGF Oligonucleotide Analog 2

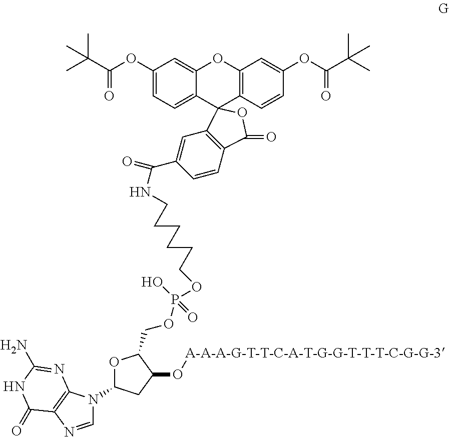

To produce 5′-(6-FAM)-VEGF Oligonucleotide Analog 2, the product of Step 1 was reacted with carboxyfluorescein NHS ester in a suitable solvent. After reverse phase chromatography using a suitable water:acetonitrile gradient the title compound 5′-(6-FAM)—VEGF Oligonucleotide Analog 2 was isolated.

### Example 3

### Preparation of Unilamellar Liposomes

Liposome composition was made up from 1,2-distearoyl-sn-glycero-phosphocholine monohydrate (DSPC):cholesterol (Chol) 55:45 molar ratio. The lipid mixture (40 mg) was dissolved in chloroform:methanol (3:1 v/v) in a round bottom flask. Organic solvents were evaporated under nitrogen using rotary evaporation and a thin phospholipid film formed along the walls of the flask. Residual solvent was removed by placing the flask in a vacuum oven under full vacuum at room temperature overnight. The resulting lipid film was hydrated by adding an ammonium sulfate solution (250 mM ammonium sulfate solution, 1 mL) to the round bottom flask and rotating the flask on a rotovap at 60° C. (without vacuum) for 30 minutes or until all the materials have dissolved. The resulting solution was diluted by addition of ammonium sulfate solution (9 mL). Multi-lamellar vesicles were extruded through 800, 400 and 100 nm pore size polycarbonate filters using a Lipex stainless steel extruder. Mean size and size distribution of liposomes were evaluated using light-scattering experiments but generally this procedure produces liposomes of 100 nM nominal diameter.

### Example 4

### Thermal Insertion of 5′-DSPE-PEG (3400)-S—C₆H₁₂—VEGF Oligonucleotide Analog 1 in Liposome prepared in Example 3 to produce PEG (3400)-S—C₆H₁₂—VEGF 1 Liposome

5′-DSPE-PEG (3400)-S—C₆H₁₂—VEGF Oligonucleotide Analog 1 may be inserted into the liposomes formed in Example 3 using the following procedure. The final extruded liposome solution prepared in Example 3 is heated to 65° C. with gentle stirring. 5′-DSPE-PEG (3400)-S—C₆H₁₂—VEGF 1 (MW 9972.0, 4.0 mg, 2.0 mole percent) is dissolved in ammonium sulfate solution (250 mM ammonium sulfate solution, 1 mL) and added to the liposome solution. At this point the solution is allowed to cool to 55° C. and a reaction is carried out at this temperature for at least 30 minutes. The reaction mixture is allowed to cool to room temperature (RT), and the particle size is determined by light-scattering techniques.

To obtain liposome bound 5′-DSPE-PEG (3400)-S—C₆H₁₂—VEGF 1 free of starting material, the reaction mixture is passed over a Sepharose CL-4B column (0.05×12 in, GE Healthcare, pre-equilibrated using PBS) using PBS as an eluent (2 mL fractions). Desired liposome product is determined using high performance liquid chromatography (HPCL) and like fractions combined.

### Example 5

### Capture of 5′-(6-FAM)-VEGF Oligonucleotide Analog 2 by PEG (3400)-S—C₆H₁₂—VEGF Oligonucleotide Analog 1 Liposomes

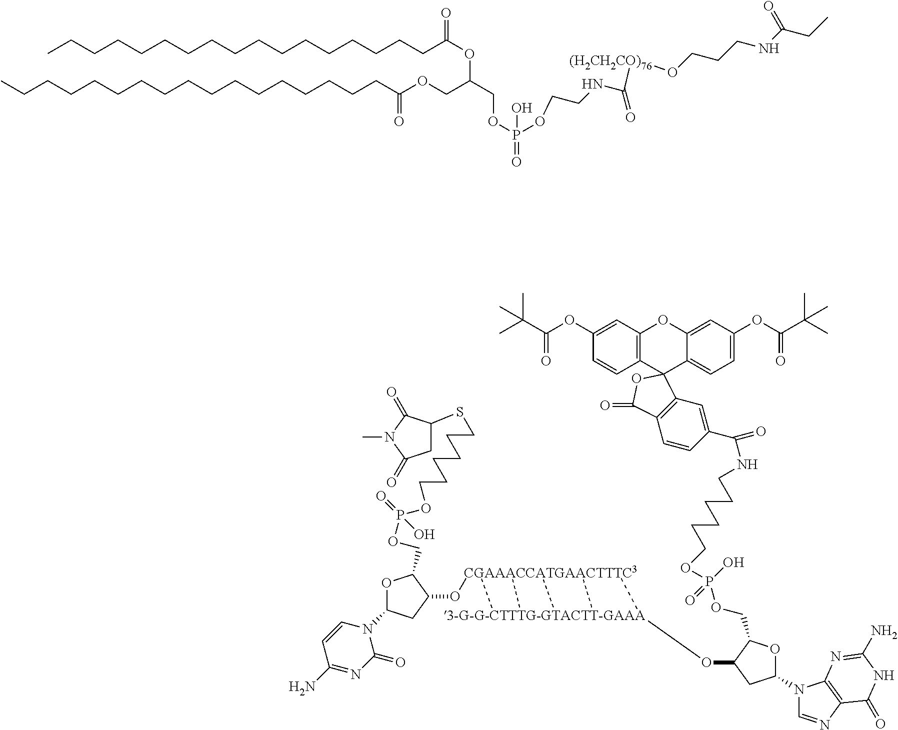

To PEG (3400)-S—C₆H₁₂—VEGF Oligonucleotide Analog 1 Liposomes in PBS from Example 3 (2 mL) is added a solution of 5′-(6-FAM)-VEGF Oligonucleotide Analog 2 in PBS (1 mg, 2×10⁻⁷ mole, in 1 mL) with swirling at RT. After 15 minutes the hybridization reaction should be essentially complete and liposomes containing the duplex DNA conjugate are separated from un-reacted single strand DNA starting material by Sepharose column chromatography as in Example 4. Analysis by HPLC using fluorescence detection will confirm the presence of ds-DNA bound fluorescein.

### Example 6

### Preparation of VEGF Oligonucleotide Analog 2, N-Succinyl-tyr-3-Octreotate

VEGF Oligonucleotide Analog 2, N-Succinyl-tyr-3-Octreotate was prepared using the following steps:

### Step 1: Preparation of N-succinyl-Tyr-3-Octreotate

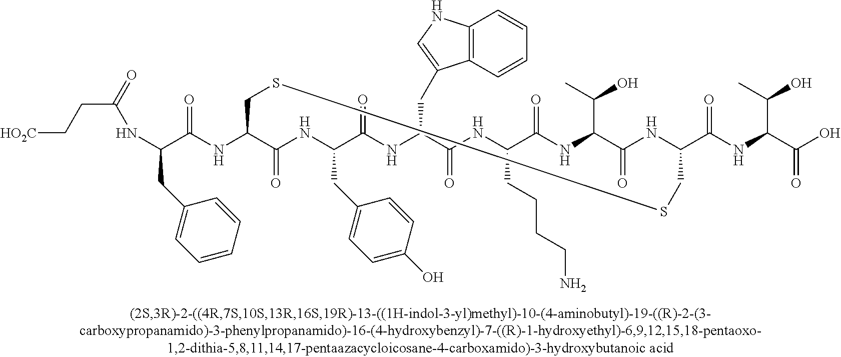 [SMILES: C[C@@H](O)[C@H](NC(=O)[C@@H]1CSSC[C@H](NC(=O)[C@@H](Cc2ccccc2)NC(=O)CCC(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

N-succinyl-Tyr-3-Octreotate, shown directly above, was prepared using standard solid support peptide FMOC synthesis techniques using extended coupling times at each step. After the peptide synthesis was complete Cys Acm protecting groups were removed and Tl(III)(TFA)₃ cyclization employed using an appropriate solvent system. The remaining protecting groups were removed and the peptide cleaved from the resin by TFA. Reverse phase HPLC (C18) showed substantially pure desired product (one peak by UV and correct MS) and this product was lyophilized and used without further purification.

### Step 2: Preparation of VEGF Oligonucleotide Analog 2, N-Succinyl-tyr-3-Octreotate

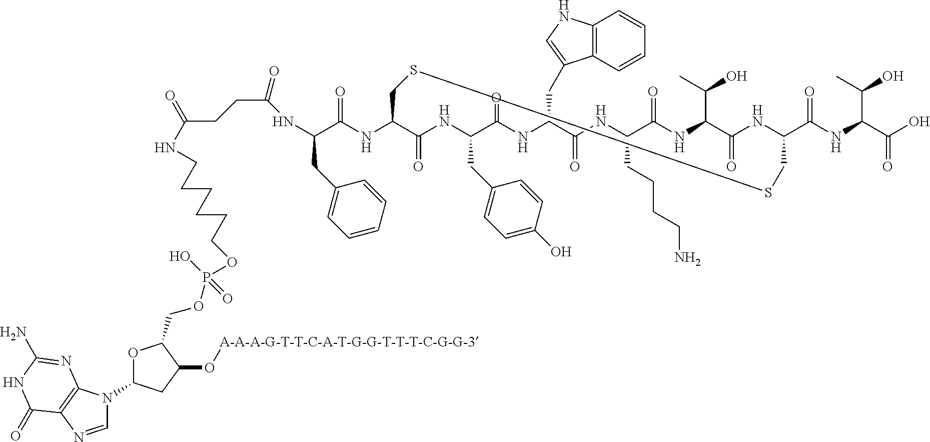

The product of Step 2 was reacted with the peptide coupling agent TBTU in a suitable solvent and converted to active ester. The mixture can be added to VEGF Oligonucleotide Analog 2 (the product of Step 1 in Example 2) dissolved in the same or similar solvent and allowed to react. Purification by reverse phase HPLC will give substantially pure VEGF Oligonucleotide Analog 2, N-Succinyl-tyr-3-Octreotate, which is the desired product.

### Example 7

### Capture of VEGF Oligonucleotide Analog 2, N-succinyl-try-3-Octreotate by PEG(3400)-S—C₆H₁₂—VEGF Oligonucleotide Analog 1 Liposome

Liposomes containing and displaying the conjugate DSPE PEG (3400) VEGF oligonucleotide analog 1 of Example 4 may be treated using substantially the quantities and conditions of Example 5 but instead substituting VEGF Oligonucleotide Analog 2, N-Succinyl-tyr-3-Octreotate for 5′-(6-FAM)-VEGF Oligonucleotide Analog 2. Liposomes are produced containing duplex double-stranded VEGF DNA with captured tyr-3-Octreotate displayed on the surface.

### Example 8

Using substantially the procedures outlined in Examples 6 and 7, components including a VEGF Oligonucleotide Analog 1 sequence can be hybridized to components including a VEGF Oligonucleotide Analog 2 sequence. For example, a VEGF Oligonucleotide Analog 2 sequence linked via a linking group to an aptamer oligonucleotide may be synthesized and purified using liquid chromatographic purification techniques. The aptamer may be an RNA-based aptamer, a DNA-based aptamer or a RNA-DNA combination-based aptamer. The purified VEGF Oligonucleotide Analog 2 sequence-linking group-aptamer can then be captured by liposomes in a similar fashion described in Example 4.

Using substantially the procedure outlined in Example 5 but substituting the VEGF oligonucleotide analog 2 sequence linked to an aptamer oligonucleotide for 5′-(6-FAM-VEGF Oligonucleotide Analog 2 gives, after purification, a liposome containing duplex double-stranded VEGF DNA with captured aptamer displayed on the surface of the liposome.

1. A targeted therapeutic or diagnostic delivery composition, comprising:
(a) a nanoparticle including a therapeutic agent or a diagnostic agent or a combination thereof;
(b) a derivatized attachment component having the formula:
A-(L¹)ₓ-C¹; and
(c) a targeting component having the formula:
C²-(L²)_{y}-T
wherein,
A is an attachment component;
each of L¹ and L² is a hydrophilic, non-immunogenic, water soluble linking group;
C¹ is one member of a preferential binding pair with a second member C², wherein C¹ and C² are oligonucleotides or oligonucleotide mimics;
T is a targeting agent; and
each of the subscripts x and y are independently 0 or 1, but at least one of x and y is other than 0;
wherein the A portion of said derivatized attachment component is attached to said nanoparticle.

2. The delivery composition of claim 1, wherein said nanoparticle is selected from the group consisting of a liposome, a micelle, a lipoprotein, a lipid-coated bubble, a block copolymer micelle, a polymersome, a niosome, an iron oxide particle, a silica particle, a dendrimer, and a quantum dot.

3. The delivery composition of claim 1, wherein said nanoparticle is a liposome selected from the group consisting of SUVs, LUVs and MLVs.

4. The delivery composition of claim 1, wherein said therapeutic agent or said diagnostic agent is embedded in, encapsulated in, or tethered to said nanoparticle.

5. The delivery composition of claim 1, wherein said attachment component comprises a functional group for covalent attachment to said nanoparticle.

6. The delivery composition of claim 1, wherein said attachment component is a lipid.

7. The delivery composition of claim 6, wherein said lipid is a phospholipid, glycolipid, sphingolipid, or cholesterol.

8. The delivery composition of claim 6, wherein the A portion of said derivatized attachment component is present in a lipid bilayer portion of said nanoparticle and, optionally said nanoparticle is a liposome.

9. The delivery composition of claim 1, wherein each of L¹ and L² is a hydrophilic, non-immunogenic, water soluble linking group independently selected from the group consisting of polyethylene glycol, polypropylene glycol, polyvinyl alcohol, polycarboxylate, polysaccharide, and dextran.

10. The delivery composition of claim 1, wherein C¹ and C² are oligonucleotides or oligonucleotide mimics of from 8-50 nucleic acids in length and C¹ is at least 70% complementary to C² across a sequence of from 8 to 30 nucleic acids and optionally, one of C¹ or C² is modified to include a linking moiety that provides covalent attachment between C¹ and C².

11. The delivery composition of claim 1, wherein C¹ and C² denature at a melting temperature between about 40° C. and about 60° C.

12. The delivery composition of claim 1, wherein C¹ and C² are from 8 to 50 nucleic acids in length and C¹ is at least 70% complementary to C².

13. The delivery composition of claim 1, wherein T is an aptamer.

14. The delivery composition of claim 1, wherein T is an aptamer that targets a site present on a receptor selected from the group consisting of MUC-1, EGFR, FOL1R, Claudin 4, MUC-4, CXCR4, CCR7, somatostatin receptor 4, Erb-B2 (erythroblastic leukaemia oncogene homologue 2) receptor, CD44 receptor, VEGF receptor-2 kinase, and nucleolin.

15. The delivery composition of claim 1, wherein each of the subscripts x and y is 1.

16. The delivery composition of claim 1, wherein x is 0 and y is 1.

17. The delivery composition of claim 1, wherein x is 1 and y is 0.

18. The delivery composition of claim 1, wherein said therapeutic agent is an anticancer agent selected from the group consisting of doxorubicin, cisplatin, oxaliplatin, carboplatin, 5-fluorouracil, gemcitibine and a taxane.

19. The delivery composition of claim 1, wherein said diagnostic agent is a radioactive agent, a fluorescent agent, or a contrast agent.

20. The delivery composition of claim 1, wherein said diagnostic agent is a radioactive agent selected from the group consisting of ¹¹¹In-DTPA, ^{99m}Tc(CO)₃-DTPA, and ^{99m}Tc(CO)₃-ENPy2.

21. (canceled)

22. (canceled)

23. A targeted delivery composition, comprising:
(a) a diagnostic or therapeutic component having the formula:
DT-(L¹)ₓ-C¹;
(b) a targeting component having the formula:
C²-(L²)_{y}-T
wherein,
DT is a therapeutic agent, diagnostic agent, or a combination thereof;
each of L¹ and L² is a hydrophilic, non-immunogenic, water soluble linking group;
C¹ is one member of a preferential binding pair with a second member C², wherein C¹ and C² are oligonucleotides or oligonucleotide mimics;
T is a targeting agent; and
each of the subscripts x and y are independently 0 or 1, but at least one of x and y is other than 0.

24. (canceled)

25. (canceled)

26. (canceled)

27. A targeted therapeutic or diagnostic delivery composition, comprising:
(a) a nanoparticle;
(b) a derivatized attachment component having the formula:
A-(L¹)ₓ-C¹; and
(c) a diagnostic or therapeutic component having the formula:
C²-(L²)_{y}-DT
wherein,
A is an attachment component;
each of L¹ and L² is a hydrophilic, non-immunogenic, water soluble linking group;
C¹ is one member of a preferential binding pair with a second member C², wherein C¹ and C² are oligonucleotides or oligonucleotide mimics;
DT is a therapeutic agent, diagnostic agent, or a combination thereof; and
each of the subscripts x and y are independently 0 or 1, but at least one of x and y is other than 0;
wherein the A portion of said derivatized attachment component is attached to said nanoparticle.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. A method of preparing a targeted therapeutic or diagnostic delivery composition, comprising contacting a derivatized attachment component having the formula:
A-(L¹)ₓ-C¹;
with a targeting component having the formula:
C²-(L²)_{y}-T
wherein,
A is an attachment component;
each of L¹ and L² is a hydrophilic, non-immunogenic, water soluble linking group;
C¹ is one member of a preferential binding pair with a second member C², wherein C¹ and C² are oligonucleotides or oligonucleotide mimics;
T is a targeting agent; and
each of the subscripts x and y are independently 0 or 1, but at least one of x and y is other than 0;
wherein the A portion of said derivatized attachment component is attached to a nanoparticle;
under conditions sufficient for a duplex to be formed between C¹ and C².

47. (canceled)

48. (canceled)

49. (canceled)

50. (canceled)

51. (canceled)

52. (canceled)

53. (canceled)

54. (canceled)


SEQUENCE LISTING (7 sequences)

SEQ ID NO: 1 - protein, 6 aa, Artificial Sequence
  synthetic His-tag, 6-His
HHHHHH

SEQ ID NO: 2 - DNA, 17 bp, Artificial Sequence
  synthetic VEGF Oligonucleotide Analog 1
ngaaaccatgaactttc

SEQ ID NO: 3 - DNA, 17 bp, Artificial Sequence
  synthetic 5′-DSPE-PEG(3400)-S-C-6H-12-VEGF Oligonucleotide Analog 1
ngaaaccatgaactttc

SEQ ID NO: 4 - DNA, 18 bp, Artificial Sequence
  synthetic VEGF Oligonucleotide Analog 2
gaaagttcatggtttcgg

SEQ ID NO: 5 - DNA, 18 bp, Artificial Sequence
  synthetic 5′-(6-FAM)-VEGF Oligonucleotide Analog 2
gaaagttcatggtttcgg

SEQ ID NO: 6 - protein, 8 aa, Artificial Sequence
  synthetic N-succinyl-Tyr-2-Octreotate
XCYXKTCT

SEQ ID NO: 7 - DNA, 18 bp, Artificial Sequence
  synthetic VEGF Oligonucleotide Analog 2, N-Succinyl-Tyr-3-Octreotate
gaaagttcatggtttcgg